(19) United States
(12) Patent Application Publication
(10) Pub. No.: US 2025/0302384 A1
(43) Pub. Date: Oct. 2, 2025

(54) A System and Method for Diagnosing Cardiometabolic Status and Prescribing Exercise for Improving Cardiometabolic Health and Fitness

(71) Applicant: NVMCO Group LLC, Greenwood Village, CO (US)

(72) Inventors: Navin Atmaram, Aurora, CO (US); Bryan Daniel Jasker, Hudson, IL (US); Izabela Slowikowska, Centennial, CO (US)

(73) Assignee: NVMCO Group LLC, Greenwood Village, CO (US)

(21) Appl. No.: 18/863,232

(22) PCT Filed: May 9, 2023
(86) PCT No.: PCT/US2023/066772

§ 371 Date: Nov. 5, 2024

Related U.S. Application Data
(60) Provisional application No. 63/340,232, filed on May 10, 2022.

(51) Int. Cl. A61B 5/00; A61B 5/0205; A61B 5/024; A61B 5/107; A61B 5/145; G16H 10/60; G16H 20/30

(52) U.S. Cl. CPC A61B 5/4866; A61B 5/0205; A61B 5/024; A61B 5/107; A61B 5/14546; A61B 5/7271; A61B 5/7475; G16H 10/60; G16H 20/30

(57) ABSTRACT

The present application provides a comprehensive approach to improving cardiometabolic health and fitness through assessing key current health and fitness metrics, testing, and assignment of appropriate movement protocols that are specific to the needs of the individual. The present application provides a unified system for cardiometabolic health testing, activity and exercise monitoring, and user education and engagement. This platform may also be useful for the management of current health dysfunction and for the prevention of disease.

### FIELD OF INVENTION

The present application generally relates to methods and systems to diagnose the cardiometabolic health and fitness of an individual through movement-based protocols and to provide guidance for improved cardiometabolic health and fitness through physical activity and exercise progressions, and feedback for improved health, fitness, and longevity.

### BACKGROUND OF INVENTION

Metabolism is the systemic integration of anabolic and catabolic physiology accounting for growth, maintenance, repair, and death. One of the main functions of metabolism is processing the macronutrients that we consume and transforming them into energy sources that the body can utilize at a cellular level to maintain the body structure and function. The glucose, amino acids, and fatty acids that result from processing the macro nutrients are exchanged for ATP (adeno sine triphosphate). ATP is then used by the body for many functions including promoting muscle contractions.

Skeletal muscle plays the primary role in metabolism and movement including influence of other organs and organ systems. Mitochondria within muscle cells (Type I>Type 2) determine fat oxidative capacity, carbohydrate oxidative capacity, and more specifically, lactate oxidative capacity. Most other major tissues create or utilize lactate as fuel including the heart and brain. Lactate can also be recycled back to glucose via the Cori Cycle in the liver. The efficiency of this process in the body may be an indicator of overall health and wellness.

Metabolic flexibility is known as the capacity of the body to switch fuels sources, such as switching between using fats and carbohydrates, to meet body's supply/demand needs (e.g., between fasting and feeding and between rest and exercise). More broadly, metabolic flexibility refers to the body's ability to make physiological adaptations. In contrast, when the body has metabolic inflexiblity, or is unable to efficiently or effectively switch between available energy systems, this is an indication of metabolic dysfunction. A lack of metabolic flexibility and greater dependence on glycolytic metabolism vs oxidative mechanisms can contribute to higher risks of developing metabolic syndrome and associated diseases, such as obesity, diabetes, heart disease, and other chronic illnesses. Lactate is the fulcrum of metabolism between glycolysis and oxidation and therefore provides an observable metric for analyzing cardiometabolic health and fitness.

Lactate (e.g., L-Lactate) is well-known as the endpoint of glycolysis. When properly absorbed by the body, lactate can be converted and used as a fuel source for the skeletal muscle, heart muscle, and the brain, with or without the requirement of oxygen. The first lactate threshold is the level at which the intensity of the exercise or stress on the body causes the lactate to accumulate in the blood at a faster rate than it is being removed by the body.

Cardiopulmonary exercise testing (CPET) through gas exchange measurement, i.e., VO₂ max, has been the gold standard method of exploring human physiology. Events consistent with physiological thresholds or turnpoints, can be identified with CPET as Ventilatory Threshold 1 (VT1) and Ventilatory Threshold 2 (VT2) or with non-CPET methods, such as with blood lactate analysis, as Lactate Threshold 1 (LT1) and Threshold 2 (LT2). LT1 is associated with the beginning of a transition from predominately fat oxidation to predominately carbohydrate metabolism, including a bias toward glycolysis (or systemic metabolic stress/strain) that can vary based on an individual's current health and fitness status. LT2 is associated with minimal to negligible fat oxidation. Cardiopulmonary exercise testing effectively describes the relationship between O2 and CO2 yet requires expensive lab equipment with substantial testing space requirements and therefore offers limited accessibility. In contrast, blood lactate testing is relatively inexpensive by comparison, requiring minimal space, and thus is more feasibly scalable for use in identifying similar physiological and metabolic turnpoints as those provided in CPET.

Blood lactate (or lactate from other origins such as sweat, breath, or urine) has historically been used in sports performance and in critical care health care settings while underutilized within the general population. The method and system herein describe an application in which blood lactate (or lactate from other origins such as sweat, breath, or urine) is used to determine cardiometabolic health and fitness of an individual, whereby the lactate measurements are used as an alternative marker of metabolism and cardiometabolic fitness compared to traditional methods of using VO₂ max to determine Cardiorespiratory Fitness (CRF).

Although numerous methods for determining an LT1 and an LT2 have been developed, to date, no process has been determined to be superior. The determination of LT1 and LT2 affords an ability to identify the oxidative capacity and maximal metabolic steady state of an individual. Additionally, the identification of these lactate thresholds can be used to determine exercise training intensities and individualized exercise prescriptions or recommendations to improve cardiometabolic fitness.

There is substantial evidence supporting a relationship between metabolic health and level of cardiorespiratory fitness. There is an independent, inverse dose-response relationship between higher CRF and non-communicable disease incidence, all-cause mortality, cardiovascular disease mortality and morbidity. Conversely, a lower CRF increases susceptibility to communicable and non-communicable diseases. There is also evidence that CRF modulates body mass index (BMI) or obesity where higher fitness yields reduced morbidity and mortality relative to all BMI levels. These relationships between metabolic health and level of fitness support the importance of activity and exercise to long-term health maintenance.

Currently, there exist no comprehensive, cohesive protocols or platforms (aka end-to-end solutions) to test, monitor, and improve cardiometabolic health and fitness. There are merely piecemeal diagnostics such as fasting blood panels, lipid panels, insulin markers, blood pressure, BMI, body composition, waist circumference, CPET, and non-medical based or non-holistic approaches. The current diagnostic approaches are best suited for identifying existing issues, but do little to anticipate or account for problems in the foundations of physiology and metabolism related to inactivity and sedentary behaviors and provide effective solutions. Further, the current approaches to management of various chronic diseases and conditions resulting from metabolic syndrome such as prescription medications and metabolic surgery can be invasive and cause additional side effects. Generalized nutritional advice and exercise recommendations may be ineffective because they are based on public health methodologies and do not provide specific guidance for a person. Therefore, even if taken in aggregate, current methods do not provide safe, effective, comprehensive, and customized movement-based diagnostics and prescriptive recommendations based on an individual's unique physiology.

Specifically, when it comes to improving health through movement and fitness, while existing approaches may collect some user data to help provide insight into the health of an individual and to offer exercise and fitness recommendations, the current models generally rely on inputs such as sex, age, height, weight, and BMI. These inputs are used to provide general assessments about the health of the individual, compared to group means of sampled data, and may provide a basic or generic exercise program to help reach generalized fitness goals. Programs based on generalized information and categories of data do not provide specific, actionable insights tailored to the unique physiology of the individual. For example, because BMI is derived based on a person's height and weight, a very overweight and deconditioned person may have the same BMI as a physically conditioned powerlifter because they may have a similar height and weight. Because of this, it is difficult to utilize this information alone to identify and address the specific needs of a person's body.

Therefore, novel testing and evaluation protocols are needed to provide customized insights to individuals regarding their current cardiometabolic health and fitness and to provide a customized, adaptable programs to assist the individual improve his or her health through physical activity and exercise.

### SUMMARY OF INVENTION

The present application provides a comprehensive approach to improving cardiometabolic health and fitness through movement analytics, protocols, and education. The present application provides a unified system for cardiometabolic health and fitness testing, activity and exercise monitoring, and user education and engagement. This platform may also be useful for the management of current health dysfunction and for the prevention of and management of diseases and conditions.

The present application provides for a movement-based assessment of cardiometabolic function (or dysfunction) as well as a method to improve cardiometabolic health and fitness through individualized and progressive, movement-based protocols. The present systems and methods provide various means to test and track various biometric and health-related data, as well as to provide customized movement-based test, including progressive protocols based on feedback cycles, that allows users to monitor and improve their cardiometabolic health and fitness. The present approach is systematic, customized to the user, and provides feedback and recalibration cycles for sustainability and progressive improvement of cardiometabolic health and fitness.

The present methods and systems allow for measuring and tracking progress in cardiometabolic health and fitness through the use of testing protocols and exercise protocols, that create feedback cycles based on an individual's changing state of cardiometabolic health and fitness.

The present methods and systems allow for monitoring and quantifying a user's heart rate recovery after periods of performing higher intensity exercise as well as to quantify acute and chronic changes in chronotropic competence (or incompetence). The present methods and systems use exercise-based diagnostic processes in which a lactate biomarker (or other biomarker deemed similar for this purpose) is used as a proxy for quantifying cardiometabolic health and fitness and indirectly characterizing mitochondrial health, capacity, or function. An objective index has been developed based on the performance of the lactate during the exercise diagnostic. The index serves as a proxy for substrate (fat) oxidation (or oxidative phenotyping) and mitochondrial capacity.

The present application provides for safe testing protocols, in which an individual performs physical activity working up to a sub-maximal effort. This approach contrasts with other methods that require users to exert maximal effort when testing (e.g., VO₂ max). Testing at a lower level of effort allows for the application of testing and exercise protocols for use with a general population, including more deconditioned or at-risk individuals that would not otherwise be able to participate in the more intensive testing methods. As additional safety precautions, the present methods and systems provide multiple screening protocols that have been incorporated throughout, including pre-test screening, monitoring physiological responses such as heart rate, heart rate recovery rate, and lactate clearance throughout the test and exercise protocols, as well as progressing, regressing, or ending testing or exercise as needed according to the individual's current level of health as indicated by measuring his or her biometric data. The constant feedback of data allows for progression and regression based on an individual's observed cardiometabolic stress or strain response thereby promoting dynamic and sustainable exercise programs that better allow for long-term health benefits.

The present application relates to a method and system for using biomarkers, such as blood lactate or other suitable biomarkers, for assessing cardiometabolic health and fitness and providing a customized protocol to guide the individual to improve cardiometabolic health and fitness. While the use of blood lactate is known, the ability to apply the blood lactate markers to a prescribed movement-based activity for the purpose of identifying and improving cardiometabolic health and fitness is novel and not previously known.

The current methods and systems involve testing and retesting the lactate in the body as the person works at various progressive levels of exercise intensity. This testing cycle allows for the recalibration and reassessment of the body's cardiometabolic health and fitness. This method has advantages over existing and previously developed methods. For example, age-based equations are static representations and do not allow additional data comparisons until a new age is achieved. Age-based analyses also assume physiological decline based on aging or age progression. These population-based derivations do not sufficiently account for unique attributes of an individual's health and health maintenance routine.

The current methods and systems identify an individual's cardiometabolic health and fitness status, also referred to as the oxidative capacity of the individual. The methods involve engaging the individual in sub-maximal exercise as determined by the individual's specific physiological parameters such as weight, BMI and blood pressure. In one embodiment, using samplings of capillary blood lactate, taken at timed intervals, provides guidance for the individual participating in various testing protocols. The blood lactate of the individual is measured concurrently with other testing markers such as power output (or speed, velocity, etc.) and heart rate throughout the novel testing protocol to inform exercise progressions or regressions based on the observed metrics.

The methods and systems described herein provide for the testing the cardiometabolic fitness of a user, comprising measuring or collecting pre-test biometric data and pre-test health data from the user, inputting the pre-test biometric data and the pre-test health data into a processing system, where the processing system assigns the user a cardiometabolic test protocol based on the pre-test biometric and pre-test health data. In the methods and systems provided, the user begins the assigned cardiometabolic test protocol, at the assigned initial work output level, for a defined interval period. Various cardiometabolic test data is collected or measured at each defined interval period and input into the processing system. The data is processed and the user is assigned a next work output level for the next defined interval period. At each defined interval period, cardiometabolic test data is measured or collected and input into the processing system, and the individual is assigned a next work output, that is higher than the previous level. This process is repeated until the processing system indicates the user has completed the cardiometabolic test protocol based on the combination of cardiometabolic test data.

At the completion of the cardiometabolic test protocol, the novel methods and systems herein provide for using data collected during the cardiometabolic test protocol to assess the cardiometabolic health and fitness of the individual. Specifically, calculating cardiometabolic fitness of a user comprises measuring or collecting the BMI of the user and inputting the BMI into a processing system, measuring or collecting the resting lactate concentration, wherein the resting lactate concentration is measured prior to the start of physical activity and inputting the measured resting lactate concentration into the processing system. Initiating physical activity at an assigned cardiometabolic fitness test protocol and increasing a power output of the physical activity over time as provided by the cardiometabolic fitness test protocol in combination with the measured a cardiometabolic test data, including heart rate and lactate, and wherein the power output of the physical activity is measured in watts. Continuing to measure the power output in watts, heart rate and the lactate concentration at each set interval and inputting the data into the processing system. Processing the power output in watts, heart rate and the lactate concentration at each interval to identify the power output in watts at the first lactate threshold (LT1). The processing system provides a data output equal to the power output in watts at LT1, divided by the BMI and multiplied by a factor of 10.

The methods and systems described herein allow for an improved ability to provide individualized exercise protocols for effective exercise dose-response based on the behavior of the blood lactate biomarker during structured physical activity. Although using blood lactate to inform certain training protocols is common in performance sports, observing blood lactate behavior in the general population during low-watt, graded, sub-maximal exercise testing protocols las not been known. The behavior of blood lactate at various exercise intensity levels can be observed and this data then be used to approximate the energy system that the body is using or fuel at the various intensity levels. used to structure the intensity of exercise or physical mnovement.

In the present methods and systems, the behavior of the blood lactate of an individual under specific conditions can be used to calculate the ideal heart rate to maintain during any exercise protocol. This is different from current methods and systems that provide generalized guidance based on broad categories such as age and other estimates, and not based on the specific cardiometabolic health and fitness status of the individual as indicated through the individual's ability to metabolize lactate in the blood.

In the present methods and systems, assigning cardiometabolic steady state heart rate zones, comprise testing the cardiometabolic fitness of a user at an assigned a cardiometabolic test protocol, wherein the cardiometabolic test protocol may be a low cardiometabolic test protocol, a middle cardiometabolic test protocol, or a high cardiometabolic test protocol, collecting cardiometabolic test data and inputting collected cardiometabolic test data into a connected processing system at each defined interval during the cardiometabolic test protocol until the user completes the cardiometabolic test protocol. The processing system assigns a user a steady state zone heart rate zone based on the combination of the cardiometabolic test protocol the user completes and the cardiometabolic test data collected during the cardiometabolic test protocol and input into the connected processing system.

In the present methods and systems, assigning cardiometabolic interval training zones, comprises testing the cardiometabolic fitness of a user at an assigned a cardiometabolic test protocol, wherein the cardiometabolic test protocol may be a low cardiometabolic test protocol, a middle cardiometabolic test protocol, or a high cardiometabolic test protocol, collecting cardiometabolic test data and inputting collected cardiometabolic test data into a connected processing system at each defined interval during the cardiometabolic test protocol until the user completes the cardiometabolic test protocol. The processing system assigns a user one or more interval training heart rate zones, wherein the interval training zone may include one or more of a light intensity interval training (LIIT), a moderate intensity interval training (MIIT), a high intensity interval training (HIIT), and a sprint intensity interval training (SIIT) based on the combination of the cardiometabolic test protocol the user completes and the cardiometabolic test data collected during the cardiometabolic test protocol and input into the connected processing system.

In the present methods and systems, adapting an interval training protocol comprises testing a cardiometabolic fitness of a user and inputting measured a cardiometabolic test data into a processing system and assigning the user an interval training protocol based on the combination of cardiometabolic test data. The interval training protocol provides a heart rate training zone for a lower intensity physical activity and one or more heart rate training zones for a higher intensity physical activity for the user to alternate between during the interval training. The interval training session also comprises a warm up in a steady state heart rate zone before the interval training session and a cool down in the steady state heart rate zone at the completion of the interval training session. The user is assigned a number of intervals to complete during each interval training session, wherein one complete interval comprises, the user working at the heart rate training zone for the lower intensity physical activity and then increasing the heart rate to work at the heart rate training zone for the higher intensity physical activity and then decreasing the heart rate back to the heart rate training zone for the lower intensity physical activity. The user alternates between heart rate zones to complete the number of intervals for the interval training session. The user may be assigned to a minimum number of interval training sessions and a maximum number of interval training sessions that should be completed in a week, wherein there is at least a 24-hour recovery period between each interval training session. The interval training protocol may be provided to a user via a user application on a connected or connected device, such as a mobile phone or tablet. The connected device is also connected to a heart rate monitor to measure the user's heart rate during each interval training session and to track the user's adherence to the number of interval training sessions completed in the week and the number of intervals completed in each interval training session using the connected device and the heart rate monitor connected to the connected; modifying the interval training protocol based on the user adherence to the number of interval training sessions completed in the week and the number of intervals completed in each interval training session in the week. The initial interval training protocol assigned to the user based on the cardiometabolic test may be progressed over time to increase the number of interval training sessions in the week, or the number of intervals may be progressed to increase the number of intervals completed during each interval training session, or the initial interval training protocol may be regressed over time to decrease the number of interval training sessions in the week, or the number of intervals may be regressed to decrease the number of intervals completed during each interval training session.

In the present methods and systems, adapting an interval training protocol in real-time, comprises assigning the user an interval training protocol and an assigned a number of intervals to complete during each interval training session, wherein one complete interval comprises, the user working at the heart rate training zone for the lower intensity physical activity and then increasing the heart rate to work at the heart rate training zone for the higher intensity physical activity and then decreasing the heart rate back to the heart rate training zone for the lower intensity physical activity. The user alternates between heart rate zones to complete the number of intervals for the interval training session. The interval training protocol may be provided to a user via a user application on a connected device, such as a mobile phone or tablet, wherein the connected device. The connected device is also connected to a heart rate monitor to measure the user's heart rate during each interval training session including the heart rate at the higher intensity physical activity and the heart rate at the lower intensity physical activity and transmitting the measured heart rates to the connected device, wherein the connected device measures the time in for the heart rate to recover from the higher intensity physical activity to down to the heart rate of the lower intensity physical activity. The heart rate for the higher intensity physical activity may be modified if the heart rate is below 15 bpm for two consecutive interval periods. The interval training heart rate for the higher intensity zone may regress from a sprint intensity interval training (SIIT) to a high intensity interval training (HIIT), from high intensity interval training (HIIT) to a moderate intensity interval training (MIIT), from a moderate intensity interval training (MIIT) to a steady state training zone, continuing to monitor the heart rate recovery and regress down, if needed, until the assigned number of intervals is complete or the interval training protocol cannot be regressed further.

The methods and systems described herein allow for an improved ability to provide individualized exercise protocols for effective exercise dose-response based on the behavior of the blood lactate biomarker during structured physical activity. Although using blood lactate to inform certain training protocols is common in performance sports, observing blood lactate behavior in the general population during low-watt, graded, sub-maximal exercise testing protocols has not been known. The behavior of blood lactate at various exercise intensity levels can be observed and this data can then be used to approximate the energy system that the body is using for fuel at the various intensity levels. used to structure the intensity of exercise or physical movement.

In the present methods and systems, the behavior of the blood lactate of an individual under specific conditions can be used to calculate the ideal heart rate to maintain during any exercise protocol. This is different from current methods and systems that provide generalized guidance based on broad categories such as age and other estimates, and not based on the specific cardiometabolic health and fitness status of the individual as indicated through the individual's ability to metabolize lactate in the blood.

### BRIEF DESCRIPTION OF FIGURES

In part, other aspects, features, benefits and advantages of the embodiments will be apparent with regard to the following description, appended claims and accompanying drawings wherein:

FIG. 1 provides a flow chart indicating the various pre-test screening factors and the use in determining the initial testing protocol.

FIG. 2 provides an exemplary progression flow from an initial low testing protocol to a middle testing protocol.

FIG. 3 provides exemplary progression or regression flow from an initial middle protocol.

FIG. 4 provides an exemplary regression flow from an initial high testing protocol to middle testing protocol.

FIG. 5 provides an illustration of the novel MetFlex Index and approximate cardiometabolic health and fitness spectrum.

FIG. 6 provides a decision tree for determining the appropriate assignment of exercise intervals for an individual testing for the first time.

FIG. 7 provides a decision tree for determining the appropriate assignment of exercise intervals for an individual that has previously tested.

FIG. 8 provides a general overview of a testing system.

FIG. 9 provides a general overview of the connection of a testing system and a user application system.

FIG. 10 provides a system flow inputting user data and initial protocol selection.

FIG. 11 provides an exemplary system flow of the testing input and output determining real-time test progression or regression.

FIG. 12 provides an exemplary system flow of the input and output determining the heart rate recovery at the completion of the testing protocol.

FIG. 13 provides exemplary flow of a SMART Zone workout provided to the user in a user application.

FIG. 14 provides exemplary flow of a basic interval workout in the user application (without regressions).

It is to be understood that the figures are not drawn to scale unless so indicated. Further, the relationship between objects in a figure may not be to scale and may in fact have a reverse relationship as to size unless so indicated. The figures are intended to bring understanding and clarity to the structure of each object shown, and thus, some features may be exaggerated in order to illustrate a specific feature of a structure. Accordingly, the figures are not intended to limit the scope and breadth of the appended claims.

### DETAILED DESCRIPTION

Various examples of the disclosure are discussed below. While specific implementations are discussed, it should be understood that these are exemplary embodiments and a person skilled in the relevant art will recognize that other components and configurations can be used without departing from the spirit and scope of this disclosure.

It should also be understood at the outset that the illustrations of the disclosed devices, methods, and systems are exemplary and may be implemented using any number of techniques. The disclosure should in no way be limited only to the illustrated implementations, drawings, and techniques provided herein, but can be modified within the scope of the appended claims along with their full scope of equivalents.

Unless otherwise specified, any use of the forms “including” and “comprising” are used in an open-ended fashion and thus should be interpreted to mean “including but not limited to . . . ”. The word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

Further, many of the embodiments described herein are described in terms of sequences of actions to be performed by, for example, elements of a computing device. It should be recognized by those skilled in the art that the various sequences of actions described herein can be performed by specific circuits and/or by program instructions executed by at least one processor. Additionally, the sequence of actions described herein can be an embodiment entirely within any form of computer-readable storage medium such that execution of the sequence of actions enables at least one processor to perform the functionality described herein. Furthermore, the sequence of actions described herein can be embodied in a combination of hardware and software. Thus, the various aspects of the present application may be embodied in a number of different forms, all of which have been contemplated to be within the scope of the claimed subject matter. In addition, for each of the embodiments described herein, the corresponding form of any such embodiment may be described herein as, for example, “a computer configured to” perform the described action.

### Method of Testing

The novel testing method described herein, SMART (Sustained Mitochondrial Aerobic Respiration Training) Test, provides for the following:

### Pre-Test Assessments

Prior to beginning the test, baseline information may be collected to pre-assess the individual's current cardiometabolic health and fitness levels. The individual may be provided with a standardized health questionnaire such as a PARQ, PARQ+ or any other similar or known formats aimed at determining such physical activity readiness and any risk. Alternatively, an individual may be presented other question types related to health and physical fitness or activity level that may be useful in assessing current health and fitness levels. Pre-test health data, such as these questions types, provide data points to be used in determining appropriate testing protocols and additionally may be used as a screening tool to minimize potential adverse impact resulting from participation in a movement-based testing protocol. Such data may be collected at any time prior to the start of physical movement component of the testing protocol. Any individual presenting answers that suggest they may have some risk in participating in movement-based testing or a lack of readiness for physical activity and exercise may be referred for medical clearance prior to testing.

In one embodiment, the individual provides a self-assessment of his or her current level of activity and such activity may be categorized in one of the following, exemplary categories:

| | |
|---|---|
| Sedentary | No intentional exercise or activity, sit or lie often, |
| | only movement with basic daily tasks at home and work. |
| Infrequent | Exercise and physically active occasionally, try to walk |
| | a couple times per week beyond daily tasks, use a gym |
| | membership 2-3 times per week, recreationally active. |
| Frequent | Intentional near daily exercise, walk, jog, cardio, |
| | weightlift beyond daily tasks and physically active |

If the individual does not fit either the definition for “sedentary” activity level or the definition for “frequent” activity level, the individual may be assigned the default activity level of “infrequent.” These categories provide for a self-assessment in accounting for variations in movement and consistency from day-to-day or week-to-week, and are commonly used and understood to provide general assessments and categorization of movement and activity.

In one embodiment, an individual is asked to identify if he or she has been diagnosed with any chronic health issues. This may be a “yes” or “no” response. If “yes,” the individual may also specify the diagnosis or note any medications that have been prescribed to treat such chronic health issues.

Additionally, other relevant data points may be collected, measured, observed, calculated, or otherwise provided. Demographic information such as age and sex, as well as biometric data such as height, weight, waist circumference, blood pressure, and pulse oxygen saturation level may be collected. Additionally, biometric data such BMI (body mass index), resting heart rate, and resting lactate may be collected or measured. Other health or biometric data such as HbA1C, glucose levels, LDL, HDL, or any other such metrics that may provide relevant insight into an individual's cardiometabolic health status may be collected or otherwise observed. Metrics are collected for each individual test subject. Any method or process for collecting these data may be used. For example, in one embodiment, the user may calculate BMI using an accepted equation known in the field, or a user may utilize a device or machine such as InBody® or DEXA® scan to provide such values. Heart rate may also be measured by any known and reliable method such as a wrist, arm, forearm, thigh, leg, or chest monitor. Similarly, other data may be collected, measured or observed through any known or later developed device or meter capable of returning accurate values.

For example, there are known methods of measuring lactate in the body. In one embodiment, common lactate measurements are taken with the use of capillary blood from, e.g., an earlobe or a fingertip. However, the use of other methods or means of obtaining lactate measurements are also conceived such as intravenous blood draws, samplings of bodily fluids other than blood (e.g., sweat, breath, or urine), and with the use of other methods such as wearable devices, sensors, or monitors.

Data such as these metrics and other points provided herein, including an individual's activity level and whether the individual suffers with chronic conditions are input into a processing system. Each data point is analyzed individually and in aggregate. The aggregate data is used to assign the individual to an initial testing protocol that provides the safest and most appropriate testing experience relevant to the individual's current and unique combination of health data.

### Initial Testing Protocol Selection

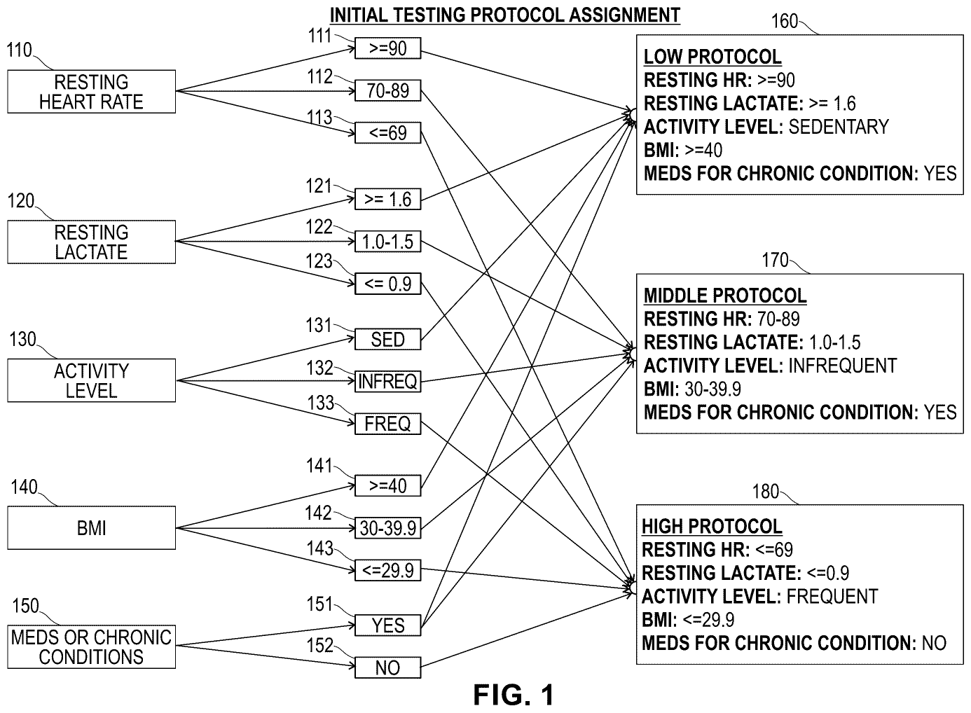

FIG. 1 presents a flow diagram for determining the initial sub-maximal testing protocol provided in the present disclosure, various sub-maximal testing protocols have been developed to guide a user's testing activity based on the specific, measured or observed states of the user. Each testing protocol is designed to provide an appropriate testing sequence based on the current state of the individual's cardiometabolic health and fitness as determined by various data points. Specifically, in one embodiment, the assignment of the initial sub-maximal testing protocol is based on the individual's unique state as determined by a combination of metrics such as BMI, resting heart rate, resting lactate, described activity level, chronic health or medication status.

In one embodiment, the resting heart rate 110 is in a range between 40 and 100 beats per minute. A heart rate monitor fitted across the chest, such as Polar® H10, or similar device and capable of accurately and continuously monitoring heart rate data and transmitting such heart rate data may be used to collect the resting heart rate and throughout the test. For the assignment of the initial testing protocol, the resting heart rate 110 will be categorized as either greater than or equal to 90 bpm 111, between 70 and 89 bpm 112, or less than or equal to 69 113.

In one embodiment, the resting lactate 120 is measured at 0.4 mmol or greater and can be measured on a lactate meter such as a Nova Biomedical® Lactate Plus Monitor (or other comparable lactate metering device). For the assignment of the initial testing protocol, the resting lactate 120 will be categorized as either greater than or equal to 1.6 mmol 121, between 1.0 and 1.5 mmol 122, or less than or equal to 0.9 mmol 123.

In one embodiment, the individual's physical activity level 130 is assessed in accordance with the exemplary definitions provided above. For the assignment of the initial testing protocol, a person's activity level 130 may be categorized as either sedentary (SED) 131, infrequent (INFREQ) 132, or frequent (FREQ) 133.

In one embodiment, the BMI 140 is determined per current World Health Organization (WHO) guidelines. The BMI may be calculated through the accepted WHO equation or measured using a device such as InBody®. For the assignment of the initial testing protocol, the BMI 140 will be categorized as either greater than or equal to 40 141, between 30.0 and 39.9 142, or less than or equal to 29.9 143.

In one embodiment, the individual will answer any question regarding chronic health issues 150 or the use of medications for treatment of such issues 150. For the assignment of the initial testing protocol, the existence of chronic conditions or use of medications to treat such conditions 150, may be categorized as either yes 151 or no 152.

In various embodiments, the measured heart rate data as observed using a heart rate monitor, the measured resting lactate data as observed using a lactate meter, the BMI as observed using a device capable of providing a BMI output, and the activity level and the use of medication or the existence of a chronic condition as reported by the user, are input into a system. The individual data points are categorized, and the combination of data provides a general profile of the user and his or her ability to handle physical activity. The holistic view of the general health and fitness of the individual determines the most appropriate starting point for the testing protocol.

In an embodiment described herein, ranges for each of the user data points are provided to correspond to each of the testing protocols. In one embodiment, a low protocol 160 includes ranges for each data point corresponding to lower levels of fitness or higher risk of potential health issues. In one embodiment, a middle protocol 170 includes ranges for each data point corresponding to middle levels of fitness or middle-range risk of potential health issues. In one embodiment, a high protocol 180 includes ranges for each data point corresponding to higher levels of fitness or lower risk of potential health issues. Exemplary low, middle, and high protocols and exemplary ranges for user data are set forth in the chart below.

| | Low 160 | Middle 170 | High 180 |
|---|---|---|---|
| | 5-Watt Protocol | 15-Watt Protocol | 30-Watt Protocol |
| Resting HR (bpm) | 90+ | 70-89 | less than or equal to 69 |
| Resting Lactate | greater than or equal to 1.6 | 1.0-1.5 | less than or equal to 0.9 |
| (mmol) | | | |
| Activity Level | Sedentary | Infrequent | Frequent |
| BMI | 40 or greater | 30-39.9 | 29.9 or less |
| Meds/Chronic | Yes | Yes | No |
| Condition | | | |

Each user is assigned to an initial testing protocol (low, middle, or high) based on the specific user data provided or measured. All user data does not have to be within the same testing protocol for the user to be assigned to a given protocol. In one embodiment, if the individual has three or more of the five metrics falling within the parameters of a certain protocol, then the individual will be assigned to the protocol in which three or more of the five criteria fall. If there is a split between two different protocols (i.e., two data metrics fall within one protocol, two data metrics fall within a second protocol, and 1 data metric falls within a third protocol), the individual would default to testing in the lower of the two protocols. As an example, if two data points indicated the middle protocol and two data points indicated the high protocol, the individual would test in the middle protocol. As another example, if two data points were in low protocol and two data points were in middle protocol, the individual would test in low protocol. In one embodiment where there is a split between the low and the high protocol (i.e., two data points were in the low protocol and two data points were in the high protocol), then the individual will begin a test at the middle protocol.

Each testing protocol—low, middle, high—provides a sub-maximal testing progression for an individual to follow as he or she moves through the testing protocol. The low testing protocol is most appropriate for a person that may be more deconditioned due to physical inactivity or who has already presented with health concerns. The low protocol starts with requiring the user to generate less physical power output (e.g., measured by the watts the user generates while pedaling on an exercise bike) and requires a slower incremental increase in the power output that the individual needs to produce during the testing process. This method of starting at a lower level and increasing more slowly is intended to minimize the stress and load on the body and to best identify LT1 if lactate clearance capacity is low. In contrast, the high testing protocol is most appropriate for a person that may be more conditioned with physical activity or who has not presented with health concerns. The high protocol starts with requiring the user to generate more physical power output, (e.g., measured by the watts the user generates while pedaling on an exercise bike) and requires a greater incremental increase in the power output that the individual needs to produce during the testing process compared to the low or middle protocols. This method of starting at a higher level and increasing more quickly, while still intended to require sub-maximal effort, is intended to acknowledge a higher level of health and fitness, including a higher lactate clearance capacity, and to promote a more efficient testing process for a body more capable of performing at a higher level. The middle testing protocol is intended to meet the needs of the individuals that have a health and fitness profile that falls in between the low and high protocol.

In one embodiment, the testing protocol assigned provides a starting point for the individual to test. The testing protocol may be adjusted in real-time based on the observance of the individual's actual lactate clearance capacity within the first two sampling cycles of testing.

### Initial Protocol Assignment Examples

Example 1: Prior to testing an individual presents with a resting heart rate of 81 bpm (middle), resting lactate of 1.6 (low), activity level of frequent (high), a BMI of 28 (high), and chronic condition or medication usage is no (high). Because three of the five metrics would place the individual in the high protocol, the individual would be assigned to the high protocol to begin testing.

Example 2: Prior to testing an individual presents with a resting heart rate of 95 bpm (low), resting lactate of 1.8 (low), activity level of infrequent (middle), a BMI of 35 (middle), and chronic condition or medication usage is yes (low/middle). Because this individual has two metrics that place him or her in the low category and 2 metrics that place him or her in the middle category, the default for a split in metrics would assign the individual to the lower testing protocol. In this case, the split between low and middle would favor the low protocol and the individual would be assigned to the low protocol to begin testing.

Example 3: Prior to testing an individual presents with a resting heart rate of 95 bpm (low), resting lactate of 1.2 (middle), activity level of frequent (high), a BMI of 25 (high), and chronic condition or medication usage is yes (low/middle). In this case, the allocation of metrics between low, middle, and high would favor the middle protocol and the individual would be assigned to the middle protocol to begin testing.

### Testing Process and Protocol Progression and Regression

The cardiometabolic test data, the data collected during the cardiometabolic test protocol, is an essential component of the testing process. Proper collection and evaluation of user data metrics inform the test protocol best suited to the user's current level of health and fitness without causing undue strain to the user's body or body systems.

Prior to the start of the physical testing process, in one embodiment, the user properly secures a heart rate monitor to his or her body. The heart rate monitor should be appropriately positioned to provide accurate and continuous feedback regarding the individual's heart rate throughout the test. In an exemplary embodiment, the heart rate monitor may be secured around the individual's chest and having appropriate contact with the user's skin and in accordance with the specific guidelines for usage of the heart rate monitoring device. Various embodiments allow for the heart rate monitor to transmit data, wirelessly or otherwise, to a connected device or system, such as, for example, a computer, tablet, or mobile device, capable of sending data to and receiving data from the heart rate monitor and capable of sending data to and receiving data from other connected devices and systems.

The physical test may be conducted using any apparatus allowing for controlled and measured power output from the individual. In one embodiment a stationary bike (e.g., upright or recumbent bike) is used while in another embodiment a treadmill may be used. In various embodiments, the individual can see the power output (or other indicator of an external load, i.e. velocity or rate of force) that he or she is working at on the apparatus. In various embodiments, the apparatus can transmit data, wirelessly or otherwise, regarding the power output level that the individual is working at, to a connected device or system capable of sending data to or receiving data from the apparatus. In an embodiment, data may be transmitted between the heart rate monitor and the apparatus (e.g., the apparatus may receive heart rate data from the heart rate monitor). In various embodiments, the heart rate monitor, and the apparatus send data to or receive data from the same connected device or system. Once an individual has a heart rate monitor and apparatus that is appropriate and capable of use in the test, the individual may begin the assigned testing protocol.

In an exemplary embodiment, all inputs are monitored via an application on a tablet or device that allows for manually populating data and automatically collecting data inputs transmitted from various connected devices. For example, the heart rate data collected from the heart rate monitor, the power output level that the individual is working at as indicated by the exercise apparatus, and the sampled blood lactate level are input or transmitted to a connected device or processing system and analyzed in combination to determine the test progression or regression for the individual at each defined interval.

The chart below illustrates the various protocols, the required starting power output (in watts), the sequential power output progression for each protocol, as well as the protocol progression or regression, if required. For each test protocol, a heart rate monitor for continuously observing the heart rate during the test protocol, an exercise apparatus for providing a means of physical activity such that the power output is measured in watts, and a means to sample an individual's lactate levels at defined intervals are needed. These data are input either manually or via transmission to a device, such as a tablet. The data is processed and based on the data, the individual may progress to higher protocol, regress to a lower protocol, or remain in his or her current testing protocol.

| | Low | Middle | High |
|---|---|---|---|
| | 5-Watt | 15-Watt | 30-Watt |
| | Protocol | Protocol | Protocol |
| Starting Power | 5 | 15 | 15 |
| Output (Watts) | | | |
| Sequential | 5, 10, 15, 25, | 15, 30, 45, 60, | 15, 30, 60, 90, |
| Watt | 35, 45, 55, 65 . . . | 75, 90, 105, | 120, 150, 180, |
| Progressions | (continues to | 120 . . . | 210 . . . |
| | increase in | (continues to | (continues to |
| | increments of 10 | increase in | increase in |
| | until test is | increments of 15 | increments of 30 |
| | complete) | until test is | until test is |
| | | complete) | complete) |
| Protocol | None | Low | Middle |
| Regression | | | |
| Protocol | Middle | High | None |
| Progression | | | |

In an embodiment, each user test begins with the individual working at the defined starting watt output level for the assigned initial protocol and progressively increases the watt output as indicated by the testing protocol. The rate of workload progression is different for each testing protocol. At a lower testing protocol, the individual increases the workload at a slower pace than at a higher testing protocol. The difference in workload progression between each protocol (i.e., low, middle, high) offers a safer testing progression for the individual without overstressing the body systems and affords more precise observation of the lactate clearance capacity during testing and specifically at transition points (e.g., LT1 and LT2).

In various embodiments, the testing process of each protocol (i.e., low, middle, high) is the same with the difference being the required power output of the user at each defined interval and the rate of increase of power output at each interval throughout the test. In various embodiments, the individual may have the lactate sampled at regular and defined intervals throughout the test. In one embodiment, blood lactate is sampled at regular three-minute intervals. Each sampled lactate value taken at the defined intervals is input into a connected device or system, such as a computer or tablet having a testing application or program, capable of accepting data inputs and administering the testing protocol as indicated by the data inputs. In an embodiment of the features presented, the individual's workload as indicated by the power output (e.g., watt level) is increased progressively until the sampled lactate level reaches a defined amount indicating metabolic stress or strain.

In an exemplary embodiment, the individual begins working at the designated watt level or power output level indicated by the assigned initial testing protocol. The lactate levels are sampled at a first and then a second defined interval. The lactate levels taken at the first and second sampling intervals after commencement of the initial testing protocol may be used to determine whether the initial testing protocol is the appropriate testing protocol for the individual to complete the test. In various embodiments, the testing protocol and corresponding workloads can either be progressed, regressed, or remain the same, based on the actual observed behavior of the lactate in the body in response to the increasing workloads performed in the initial testing protocol. The evaluation of the lactate and the real-time adjustment of the testing protocol to accommodate the actual and specific behavior of the lactate clearance capacity of the individual after the second sampling period serves as another mechanism to minimize stress to the individual's body system caused by increasing workload.

In one embodiment, the lactate sample taken at the first interval is compared to the resting lactate sample and the lactate sample taken at the second interval is compared to the lactate sample taken at the first interval. In various embodiments, the relative change in lactate level compared to the previous sample periods is indicative of the individual's cardiometabolic stress level. In various embodiments, if the individual is sufficiently clearing lactate as the workload increases, it may be determined that the individual is working in an appropriate testing protocol or that the individual may be able to perform at a higher testing protocol. In contrast, if an individual is unable to clear lactate as the workload increases, it may be determined that the individual may need to move to a lower testing protocol. In various embodiments, the testing protocol assigned after the second sampling period is the testing protocol that the user will remain in for the duration of the test.

In an exemplary embodiment, the individual pre-test biometric data and pre-test health data metrics indicate that the individual is assigned to a middle initial testing protocol. The individual begins the initial testing protocol working at the designated middle protocol starting power output. The individual begins working, e.g., pedaling the bike, to reach the desired power output level. The individual will work to produce a constant power output for the entire interval period. At the conclusion of the first designated interval, the lactate of the individual is measured and the data is recorded in the connected device or system, where the first lactate sample is compared to the resting lactate sample. The individual is directed to continue the current protocol and increase exercise to the next power output level for the middle protocol. The individual will continue to work to produce a constant power output for the second interval period. At the conclusion of the second designated interval, the lactate of the individual is measured again. The second lactate sample is recorded in the connected device or system and compared to the first lactate sample. The individual's ability to clear lactate as demonstrated during the first two testing intervals determines the protocol that the individual is assigned for the duration of the test (e.g., test progression, regression or stay in the current protocol). In various embodiments, two consecutive increases in lactate measurements may result in a regression of testing protocol and two consecutive decreases in lactate measurements may result in a progression of testing protocol. If a change in test protocol is recommended based on the behavior of the lactate measured in the first two interval periods, the initial testing protocol will be adapted to the new testing protocol. The individual will continue now periodically increase the workout based on the indications provided by the new testing protocol. The lactate will continue to be observed at each subsequent interval until the test is concluded and the maximum lactate reading is observed.

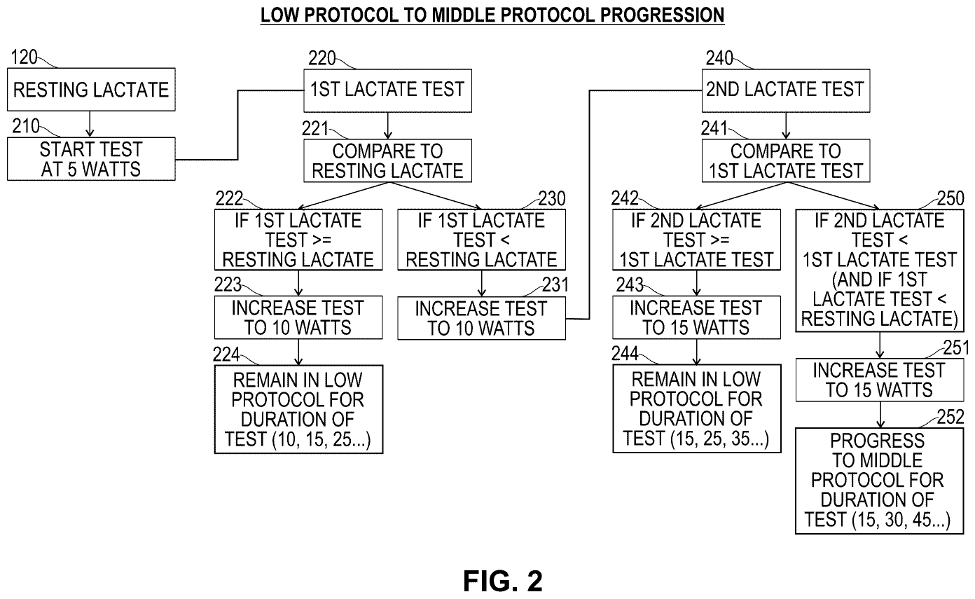

The flow diagrams of FIGS. 2-5 illustrate various specific embodiments. For example, FIG. 2 illustrates the process for evaluating progression from a low protocol to a middle protocol. Specifically, the initial resting lactate 120 may indicate that the individual starts the testing protocol at a low protocol 210. After completing the first interval, the first lactate sample 220 is measured and compared 221 to the resting lactate 120. If the first lactate 220 measure is greater than or equal to the resting lactate 120, the individual is instructed to increase the power output 223 and will remain in the low protocol for the duration of the test 224. If the first lactate sample 220 is lower than the resting lactate 120, the individual is instructed to increase the power output 231 for the duration of the second interval. At the completion of the second interval, a second lactate sample 240 is taken. The second lactate sample 240 is compared 241 to the first lactate sample 220. If the second lactate sample 240 is greater than or equal to the first lactate sample 220, the individual is instructed to increase the power output 243 and will remain in the low protocol 244 for the duration of the test. If the second lactate sample 240 is less than the first lactate sample 220 (and if the first lactate sample 220 was less than the resting lactate 120), the individual is instructed to increase the power output 251 and will progress to complete the test at the middle protocol 252. In summary, the individual would complete the first two intervals at the low protocol, allowing for the observation of two lactate samples. If there have been two consecutive decreases in the first two lactate samples as compared to each immediate prior sample, representing the individual's ability to clear lactate from the system, then the individual is progressed to the required watt output of the middle protocol in the third interval. In this example, the progressive workload in watts may be 5, 10, 15, 30, 45 . . . and continue in 15 watt increments in accordance with the middle protocol until the individual has completed his or her testing cycle. An individual cannot progress from low protocol to high protocol.

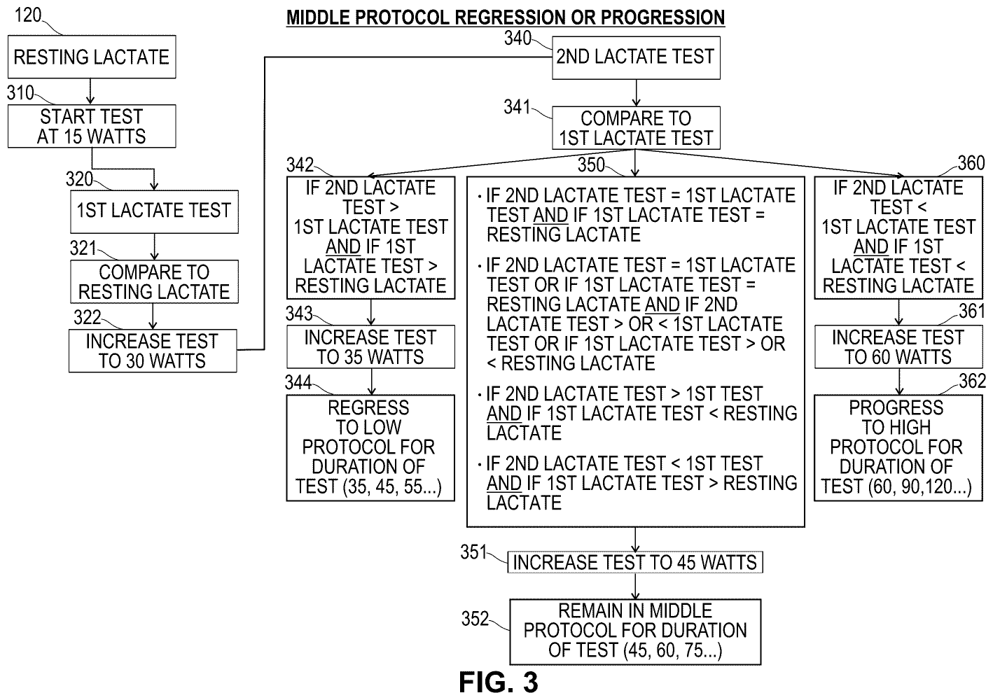

FIG. 3 illustrates the process for evaluating the progression or regression of an individual that is initially assigned to a middle protocol. Specifically, the initial resting lactate 120 may indicate that the individual starts the testing protocol at a middle protocol 310. After completing the first interval, the first lactate sample 320 is measured and compared 321 to the resting lactate 120. The individual is instructed to increase the power output 322 for the duration of the second interval. At the completion of the second interval, a second lactate sample 340 is taken. The second lactate sample 340 is compared 341 to the first lactate sample 320. If the second lactate sample 340 is greater than the first lactate sample 320 and if the first lactate sample 320 is greater than the resting lactate sample 120, then the individual is instructed to increase the power output 343 and will regress to complete the testing at the low protocol 344. If the second lactate sample 340 is less than the first lactate sample 320 and if the first lactate sample 320 is less than the resting lactate sample 120, then the individual is instructed to increase the power output 361 and will progress to complete the testing at the high protocol 362. If neither the regression nor progression analysis applies 350, the individual is instructed to increase the power output 351 and will complete the testing at the middle protocol 352. In one example, if the individual is assigned to a middle protocol and the individual is regressed to a low protocol after observing the second lactate sample, the individual would complete the first two intervals at the middle protocol, allowing for the proper observation of the consecutive increases in the first two lactate samples, and then regress to the watts of the low protocol in the third interval. In this example the progressive workload in watts may be 15, 30, 35, 45, 55 . . . and continue in 10 watt increments in accordance with the low protocol until the individual has completed his or her testing cycle. In another example, if the individual is initially assigned to a middle protocol and the individual is progressed to a high protocol after observing the second lactate sample, the individual would complete the first two intervals at the middle protocol, allowing for the proper observation of the consecutive decreases in the first two lactate samples as compared to each immediate prior sample, and then progress to the required watt output of the high protocol in the third interval. In this example, the progressive workload in watts may be 15, 30, 60, 90, 120 . . . and continue in 30 watt increments in accordance with the high protocol until the individual has completed his or her testing cycle. If an individual is initially assigned to a middle protocol, and after observing and comparing the change in lactate measure from the resting lactate sample to the first lactate sample and from the first lactate sample to the second lactate sample, and neither a regression or progression are appropriate, the individual will complete the testing at the middle protocol. In this example, the progressive workload in watts 15, 30, 45, 60, 75 . . . and continue in 15 watt increments in accordance with the middle protocol until the individual has completed his or her testing cycle.

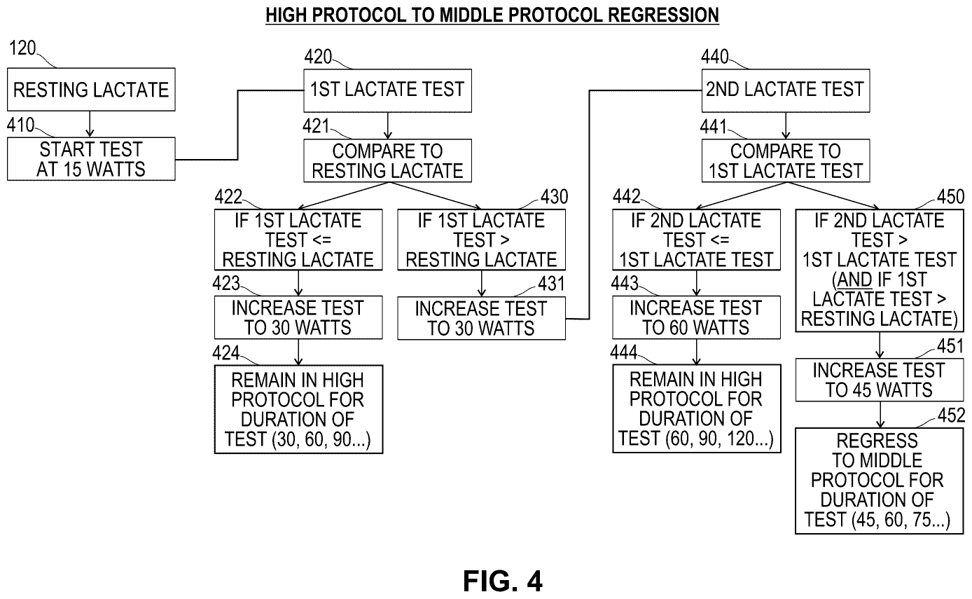

FIG. 4 illustrates the process for evaluating regression from a high protocol to a middle protocol. Specifically, the initial resting lactate 120 may indicate that the individual starts the testing protocol at a high protocol 410. After completing the first interval, the first lactate sample 420 is measured and compared 421 to the resting lactate 120. If the first lactate 420 measure is less than or equal to the resting lactate 120, the individual is instructed to increase the power output 423 and will remain in the high protocol for the duration of the test 424. If the first lactate sample 420 is greater than the resting lactate 120, the individual is instructed to increase the power output 431 for the duration of the second interval. At the completion of the second interval, a second lactate sample 440 is taken. The second lactate sample 440 is compared 441 to the first lactate sample 420. If the second lactate sample 440 is less than or equal to the first lactate sample 120, the individual is instructed to increase the power output 443 and will remain in the high protocol 444 for the duration of the test. If the second lactate sample 440 is greater than the first lactate sample 420 (and if the first lactate sample 420 was greater than the resting lactate 120), the individual is instructed to increase the power output 451 and will regress to complete the test at the middle protocol 452. In summary, if the individual is assigned to a high protocol and the individual is regressed to a middle protocol after observing the second lactate sample, the individual would complete the first two intervals at the high protocol, allowing for the proper observation of the consecutive increases in the first two lactate samples as compared to each immediate prior sample, and then regress to the watts of the middle protocol in the third interval. In this example the progressive workload in watts may be 15, 30, 45, 60, 75 . . . and continue in 15 watt increments in accordance with the middle protocol until the individual has completed his or her testing cycle. An individual cannot regress from high protocol to low protocol.

In each embodiment, even if a protocol is regressed after the second interval period, the individual's required workload increases over the previous interval period. All testing protocols are complete when the lactate level reaches a concentration of 5.9 mmol or sooner if the individual is unable to continue the assigned testing protocol.

### Testing Protocol Progression and Regression Examples

Example 1: A user was assigned to a low initial testing protocol based on his pre-test data. The user's resting lactate measured 1.1 mmol. After starting the initial testing protocol and working at the required 5-watt power output as indicated by the low test protocol for a period of 3 min, the user's blood lactate was sampled and measured 0.9 mmol. The first lactate sample was less than the resting lactate level and the user was instructed to increase his power output to 10 watts as indicated by the low test protocol. After working at 10 watts for 3 min, the user's blood lactate was sampled and measured 0.9 mmol. The second lactate sample was the same as the first lactate sample and the user was instructed to increase his power output to work at 15 watts as indicated by the low protocol and to continue increasing the power output as indicated by the low protocol until the individual completes the testing protocol.

Example 2: A user was assigned to a middle initial testing protocol based on his pre-test data. The user's resting lactate measured 1.1 mmol. After starting the initial testing protocol and working at the required 15-watt power output as indicated by the middle test protocol for a period of 3 min, the user's blood lactate was sampled and measured 0.9 mmol. The first lactate sample was less than the resting lactate level and the user was instructed to increase his power output to 30 watts as indicated by the middle test protocol. After working at 30 watts for 3 min, the user's blood lactate was sampled and measured 0.8 mmol. The second lactate sample was less than the first lactate sample and the user was instructed to increase his power output to work at 30 watts as indicated by the high protocol and to continue increasing the power output as indicated by the high protocol until the individual completes the testing protocol.

Example 3: A user was assigned to a high initial testing protocol based on his pre-test data. The user's resting lactate measured 1.1 mmol. After starting the initial testing protocol and working at the required 15-watt power output as indicated by the high test protocol for a period of 3 min, the user's blood lactate was sampled and measured 1.2 mmol. The first lactate sample was greater than the resting lactate level and the user was instructed to increase his power output to 30 watts as indicated by the high test protocol. After working at 30 watts for 3 min, the user's blood lactate was sampled and measured 1.3 mmol. The second lactate sample was greater than the first lactate sample and the user was instructed to increase his power output to work at 45 watts as indicated by the middle protocol and to continue increasing the power output as indicated by the middle protocol until the individual completes the testing protocol.

### Post-Test Recovery: Heart Rate Recovery Rate and Lactate Clearance Capacity

The individual is also observed during the cool down or recovery period post-completion of the testing protocol. In one embodiment, during this period, the individual may continue the same movement patterns that they exhibited for testing (e.g., walking, cycling, etc.) however, the individual may substantially reduce the movement intensity or completely stop the movement so that the reduced intensity allows the heart rate to begin to decrease, or otherwise recover from the increased stress of the testing protocol. In an exemplary embodiment, the movement intensity may be decreased until the heart rate reaches the new steady state as determined by the output of the testing protocol and then the individual may maintain the steady state for the remainder of the recover period. During this process, the individual's heart rate continues to be measured and may be automatically populated into a connected device or system at regular intervals for a set period of time (e.g., the heart rate is measured every minute for a 10 minute period). In one embodiment, the post-test heart rate recovery rate is calculated based on the average of the measured heart rate at the first- and second-minutes mark post-completion of the testing protocol. For example, if the heart rate measured at the completion of the test (i.e., the maximal sub-maximal heart rate), was 150, the heart rate measured at the first minute post-completion of the testing protocol was 130, and the heart rate measure at the second minute post-completion of the testing protocol was 100, then the heart rate recovery rate for this individual would be recorded as 25 bpm. This is calculated by subtracting the heart rate at the first minute post-test from the heart rate at the end of the test (e.g., 150 bpm−130 bpm=20 bpm) to determine the heart rate recovery within the first minute. Similarly, the heart rate at the second minute post-test would be subtracted from the heart at the end of the first minute post-test (e.g., 130 bpm−100 bpm=30 bpm) to determine the heart rate recovery within the second minute. The 2-minute heart rate recovery would be the average recovery over the first two minutes (e.g., 20 bpm+30 bpm=50 bpm, 50 bpm/2 minutes=25 bpm).

In one embodiment, the blood lactate level is also observed during the recovery period. The lactate level may be measured at intervals less frequent than the heart rate. In one exemplary embodiment, in a 10-minute recovery period, the lactate is measured at the fifth-minute of recovery, and again at the tenth-minute of recovery. The observed data points are input into the connected device or system and used to calculate the individual's lactate clearance capacity rate, or change in lactate accumulation over time. The recovery rate is determined as a function of the individual's ability to clear the lactate from the body post-exercise when compared to the final lactate measure at the conclusion of the recovery period. For example, if an individual had a final testing lactate measure of 6.0 mmol and the lactate sample at 5-minutes post-test was 4.5 mmol, the lactate clearance rate would be 0.3 mmol. This is calculated by subtracting the lactate measure at the 5-minutes post-test from the lactate measure at the end of the test (e.g., 6.0 mmol−4.5 mmol=1.5 mmol) and dividing the difference by the number of minutes to determine the lactate clearance rate per min (e.g., 1.5 mmol/5 min=0.3 mmol/min). Similarly, it can be noted that the individual does not clear lactate and instead, a lactate accumulation is observed. An example of lactate accumulate would be, if an individual had a final testing lactate measure of 6.0 mmol and the lactate sample at 5-minutes post-test was 7.5 mmol, in this case, the lactate measure at 5-minutes is higher than the measurement at the end of the test. This would represent a lactate accumulation rate of 0.3 mmol. This is calculated by subtracting the lactate measure at the 5-minutes post-test from the lactate measure at the end of the test (e.g., 6.0 mmol−7.5 mmol=−1.5 mmol) and dividing the difference by the number of minutes to determine the lactate clearance rate per min (e.g., −1.5 mmol/5 min=−0.3 mmol/min).

### Testing Data Output

At the conclusion of the testing protocol, the various heart rate data, power output, and lactate measurements, in combination with other collected health data, are used to assess the cardiometabolic health and fitness of the individual and to provide objective feedback to guide the individual's exercise behavior. Traditionally, lactate transition points are calculated as a function of the individual's heart rate at various points. The first lactate transition point, LT1, can be described as the lowest intensity at which there is an increase in blood lactate concentration above the resting lactate measurement or the lowest lactate value observed during early testing. The LT1 can be used to demarcate the transition between zone 1 and zone 2 in a three-zone model. The second lactate transition point, LT2, is also known as the maximal lactate steady state (MLSS) and can be used to demarcate zone 2 and zone 3 in a three-zone model. Knowledge of these transition points is not new. Further, there are numerous ways that these transition points may be calculated or approximated.

The present application leverages the understanding of lactate transition points in a novel way. Specifically, in the present application, the transition between zone 1 and zone 2, LT1, is assigned a value, depending on the testing protocol (e.g., low, middle, or high) that the individual completes. The slope intercept equation may then be used to solve for the heart rate at LT1.

$$m = \frac{Δ y}{Δ x} = \frac{y 2 - y 1}{x 2 - x 1} and x 2 = \frac{y 2 - y 1}{m} + x 1$$

In an embodiment of the novel application, if the individual completed the test at a low protocol, then 2.0 mmol lactate value is used to calculate the individual's heart rate at LT1. If the individual completed the test at a middle testing protocol, then 1.9 mmol lactate value is used to calculate the individual's heart rate at LT1. If the individual completed the test at a high protocol, then 1.8 mmol lactate value is used to calculate the individual's heart rate at LT1. Using this method approximates the historic identification of LT1 at 2 mmol and below. It follows that an individual who is less fit will have a higher resting lactate prior to the exercise test, a higher baseline lactate during exercise testing, a reduced lactate clearance capacity, and a higher threshold (LT1), whereas an individual who is more fit will have a lower resting lactate prior to the exercise test, a lower baseline lactate during exercise testing, and an increased lactate clearance capacity a lower threshold (LT1). This method also approximates other determinative measures relative to VO₂ max testing, such as an individual's oxidation capacity. Generally, a less fit person has a smaller window of oxidation due to, in part, a poorer capacity to clear lactate from the body while a more fit person has a larger window of oxidation due, in part, to a greater capacity to clear lactate.

The same slope intercept equation methodology is used to calculate heart rate at LT2 and LT3. The respective lactate values used to calculate LT2 and LT3 do not vary based on the testing protocol that the individual completed the test. To calculate LT2, 3.9 mmol lactate value is used to calculate the individual's heart rate. To calculate LT3, 5.9 mmol lactate is used to calculate the individual's heart rate.

Specifically, the present application uses the measured heart rate and lactate measurements taken at each interval during the testing protocol. If a measured data is taken during a set sampling interval such that the heart rate measurement aligns to an assigned lactate value (e.g., LT1 1.8 mmol, 1.9 mmol, or 2.0 mmol depending on the assigned testing protocol, LT2 at 3.9 mmol, or LT3 at 5.9 mmol) then the measured heart rate is used as the basis for any additional calculations. In contrast, if the measured heart rate does not align with one of the assigned lactate values, then using the measured heart rate and lactate levels for two periods, the heart rate at the assigned value is derived using the slope intercept equation wherein the measured data points are used to calculate the slope of the line and then the calculated slope is used to calculate the heart rate at the desired lactate point.

### Lactate Threshold Transition Calculation Examples

Example 1: If an individual completes a low testing protocol and has a heart rate of 124 bpm at 1.5 mmol and a heart rate of 135 bpm at 2.3 mmol, then the heart rate at LT1 (2.0 mmol) would be derived from the slope intercept equation to be 131. Specifically, the difference in the two measured lactate values would provide Δy=(2.3−1.5)=0.8 and the two measured heart rate values would provide Δx=(135−124)=11. This yields m=0.073. Now, the calculated slope, can be used to solve for the heart rate, x2 at 2.0 mmol, LT1 for low protocol. In this case, the heart rate at LT1 equals Δy=(2.0−1.5)=0.5, divided by the calculated m=0.073, plus 124. This yields a heart rate at LT1 of 131 bpm.

Example 2: If an individual completes a high testing protocol and has a heart rate of 124 bpm at 1.5 mmol and a heart rate of 135 bpm at 2.3 mmol, then the heart rate at LT1 (1.8 mmol) would be derived from the slope intercept equation to be 128. Specifically, the difference in the two measured lactate values would provide Δy=(2.3−1.5)=0.8 and the two measured heart rate values would provide Δx=(135−124)=11. This yields m=0.073. Now, the calculated slope, can be used to solve for the heart rate, x2 at 1.8 mmol, LT1 for low protocol. In this case, the heart rate at LT1 equals Δy=(1.8−1.5)=0.3, divided by the calculated m=0.073, plus 124. This yields a heart rate at LT1 of 128 bpm.

Example 3: If an individual completes a middle testing protocol and has a heart rate of 140 bpm at 3.4 mmol and a heart rate of 160 bpm at 3.9, then the heart rate at LT2 would be 160 bpm. In this case, 160 was the measured heart rate at 3.9 mmol, the assigned lactate value for LT2. The assigned protocol (e.g., low, middle, or high) does not affect the calculation of LT2.

Example 4: If an individual completes a middle testing protocol and has a heart rate of 155 bpm at 5.2 mmol and a heart rate of 175 bpm at 6.5 mmol, then the heart rate at LT3 (5.9 mmol) would be derived from the slope intercept equation to be 166. Specifically, the difference in the two measured lactate values would provide Δy=(6.5−5.2)=1.3 and the two measured heart rate values would provide Δx=(175−155)=20. This yields m=0.065. Now, the calculated slope, can be used to solve for the heart rate, x2 at 5.9 mmol, LT3. In this case, the heart rate at LT3 equals Δy=(5.9−5.2)=0.7, divided by the calculated m=0.065, plus 155. This yields a heart rate at LT3 of 166 bpm. The assigned protocol (e.g., low, middle, or high) does not affect the calculation of LT3.

### Metabolic Flexibility Index (MetFlex Index™)

Metabolic flexibility broadly refers to the body's ability to switch between the energy systems that fuel the body's activities. The MetFlex Index™ was conceived to perform as a novel indicator of Cardiometabolic Fitness (CMF) and a proxy for cardiorespiratory fitness (CRF). Cardiorespiratory fitness is customarily defined by VO₂ max, a more expensive and more strenuous testing process, of which can also be derived Metabolic Equivalents of Task (METs). It is commonly understood that less than 4 METs indicates significant dysfunction and a decline of the cardiorespiratory system, between 4-10 METs indicates progressively reduced mortality trends (i.e., as the METs increase, the likelihood of cardiorespiratory and associated dysfunctions, decrease) and greater than 10 METs indicates fitness for health and longevity. Prior to the methods provided in the present application, there is no known measurement or testing process for Cardiometabolic Fitness. The novel testing protocol provides a means to test an individual's Cardiometabolic Fitness level and further the novel MetFlex Index™ allows the testing data to be used to calculate an indexed-based assessment of the individual's relative potential for health issues related to poor cardiometabolic fitness.

The novel MetFlex Index™ may also be relevant as a risk stratification tool. The tool can be used to understand the relationship between oxidative capacity relative to power output and an individual's current BMI. The MetFlex Index™ can also be used to anticipate cardiometabolic decline prior to other traditional known markers. Specifically, the combination of the testing protocol and the novel index allow for the identification of individual's most at-risk of developing metabolic syndrome and associated disease.

The MetFlex Index™ may also be used as a surrogate marker for fat oxidation capacity in the body. The body's ability to oxidize fat begins to decline on or before the LT1 and the ability to oxidize fat is significantly reduced by LT2 and thereafter. The ability to clear lactate at higher power outputs is correlated with the ability to prolong utilization of fat oxidation, preserving glucose by delaying energy dependence of a given activity or intensity of activity on glycolysis and carbohydrate oxidation. The higher the watts generated at each lactate transition point, the greater the lactate clearance capacity. A low MetFlex score indicates a lower capacity to clear lactate and indicates poorer capacity to oxidize fat in the body. In contrast, a high MetFlex represents a higher capacity to clear lactate from the body and better capacity to oxidize fat in the body. Other exemplary observed relationships with the MetFlex Index™ include:

| Lower MetFlex Index ™ | Higher MetFlex Index ™ |
|---|---|
| Decreased use of fat as a primary | Increased use of fat as a primary energy substrate |
| energy substrate | |
| Low power output relative to body | High power output relative to body |
| mass | mass |
| Reduced lactate clearance capacity | Increased lactate clearance |
| | capacity |
| Reduced mitochondrial content and | Increased mitochondrial content |
| function | and function |
| Dependent on glucose as a primary energy substrate | Delayed need for glucose as a |
| | primary energy substrate |
| Relates to reduced CMF | Relates to increased CMF |
| Relates to decreased longevity | Relates to increased longevity |
| Relates to reduced health span | Relates to increased health span |
| Reduced cellular health | Improved cellular health |
| Relevance to BMI | Relevance to BMI |

### MetFlex Index™ (MFI) Calculation

An individual's lactate behavior at various stages during the test can be used as a proxy representation of the metabolic health and fitness of the individual. In one embodiment, the novel MFI value is calculated as a function of the watts performed at LT1 relative to BMI from the combination of measurements and data collected prior to the start of the testing protocol and during each interval period of the testing protocol and as processed by the processing system. In the current embodiment, the numerator is determined by measuring the watts the individual performs (e.g., the power output generated by the individual pedaling on the bike) during testing at LT1. The denominator incorporates the individual's BMI measurement to provide more standardized comparisons to other health metrics that utilize BMIs. Specifically, the derived value is:

$$MetFlex Index ™ = ( \frac{Watts at LT 1}{BMI} ) * 10$$

Similar to the determination of heart rate at LT1, the power output can also be derived given the slope intercept equation and two points. If provided the same data points provided in Example 1 above, with the addition of the corresponding power outputs, which would be known based on the assigned testing protocol and the individual's response to the increasing work outputs required by the testing protocol, the exemplary calculation would be as follows: an individual completes a low testing protocol and has a heart rate of 124 bpm at 1.5 mmol and 15 watts, and a heart rate of 135 bpm at 2.3 mmol and 25 watts. The heart rate at LT1 (2.0 mmol) would be derived from the slope intercept equation to be 131 and the power output at LT1 would be 21 watts. Specifically, the difference in the two measured lactate values would provide Δy=(2.3−1.5)=0.8 and the two measured power outputs values would provide Δx=(15−25)=10. This yields m=0.8. Now, the calculated slope, can be used to solve for the power output, x2 at 2.0 mmol, LT1 for low protocol. In this case, the heart rate at LT1 equals Δy=(2.0−1.5)=0.5, divided by the calculated m=0.08, plus 15. This yields a power output at LT1 of 21 watts. With a known BMI, the novel MFI can be calculated such that a correlation to cardiometabolic health and fitness can be determined.

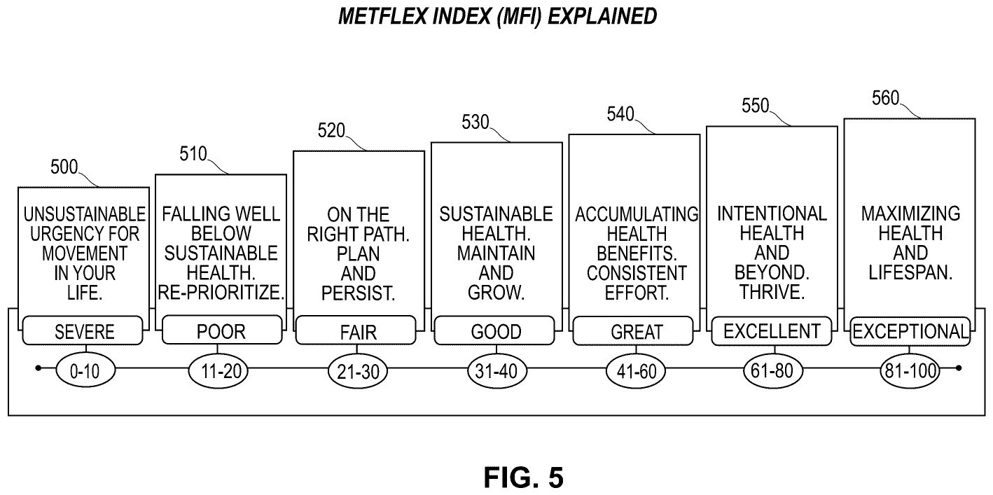

The MetFlex Index™ value provides an approximation of the oxidation capacity of the individual relative to lactate metabolism. In various embodiments, the lower the MFI value, the more likely the person is at risk of having or for developing cardiometabolic health issues and the higher the MFI value, the more likely the person is at a lower risk of or for developing cardiometabolic health issues. FIG. 5 illustrates an exemplary scale of the MetFlex Index™ values and relative meanings. In the scale, the calculated MFI values are shown in groupings such that a MFI of 0-10 500 represents someone that may have severe cardiometabolic health issues or risk of developing issues, 11-20 510 represents someone that may have poor cardiometabolic health or risk of developing issues, 21-30 520 represents someone that may have fair cardiometabolic health or risk of developing issues, 31-40 530 represents someone that may have good cardiometabolic health or at lower risk of developing issues, 41-60 540 represents someone that may have great cardiometabolic health or at lower risk of developing issues, 61-80 550 represents someone that may have excellent cardiometabolic health or at lower risk of developing issues, and 81-100 560 represents someone that may have exceptional cardiometabolic health or at lower risk of developing issues.

### Power Durability Index (PDI) Calculation

An individual's lactate metabolism at various stages during the test can be used as a proxy representation of the metabolic health and fitness of the individual. In one embodiment, the novel Power Durability Index value is calculated as a function of the watts performed at LT2 relative to BMI. In the current embodiment, the numerator is determined by the watts the individual performs (e.g., the power output generated by the individual pedaling on the bike) during testing at LT2. The denominator incorporates the BMI measurement to provide more standardized comparisons to other health metrics that utilize BMIs. Specifically, the derived value is:

$$Power Durability Index = ( \frac{Watts at LT 2}{BMI} ) * 10$$

The values needed to calculate the power durability index would be determined in a similar manner to the exemplary calculations provided previously. The power durability index value provides an approximation of the maximal sustainable cardiometabolic fitness of the individual relative to lactate behavior. In various embodiments, the lower the power durability index value, the lower the maximal sustainable power output relative to lactate behavior, relative to BMI and the higher the power durability index value, the higher the maximal sustainable power relative to lactate behavior, relative to BMI.

### Sustained Mitochondrial Aerobic Respiration Training (SMART) Zone and Exercise Protocols

The data collected during the pre-test and throughout the testing protocols including lactate measurements as measured by a lactate meter, heart rates as measured by a heart rate monitor, and the individual's power outputs as measured in watts are input into the device or system, and are processed in combination, to assess the individual's current level of cardiometabolic health and fitness as well as to provide the individual with exercise protocols based on the individual's current physiological status. The various exercise protocols that are designed to optimize the effectiveness of an individual's movement activity by accounting for the individual's actual physical capacity to process fat as a fuel source as observed by the lactate measurements, in combination with the various other testing metrics, during the testing protocol.

In one embodiment, the individual's performance in the testing protocol allows for the determination of an optimal steady state exercise heart rate zone as well as the determination of optimal interval training zones specific to the cardiometabolic fitness status of the individual at the time of testing. The steady state protocol, or SMART Zone, is the optimal exercise training zone and is provided to each individual who completes the testing protocol, or alternatively, is able to attain the minimum lactate level to calculate steady state, based on the individual's assigned testing protocol.

In one embodiment, the SMART Zone is a customized form of steady state cardiometabolic exercise that replaces the traditional “aerobic” and “cardio” exercise concepts. In the present embodiment, the SMART Zone approximates the heart rate zone that provides maximal use of fat oxidation as a fuel and to maximize lactate clearance capacity prior to additional lactate accumulation. The power generated within this SMART Zone, relative to LT1, is a component of the MetFlex Index.

| | Low | Middle | High |
|---|---|---|---|
| | 5-Watt Protocol | 15-Watt Protocol | 30-Watt Protocol |
| Max steady state | HR @ 2 mmol | HR @ 1.9 mmol | HR @ 1.8 mmol |
| heart rate | | | |
| (SMART Zone max) | | | |
| Min steady state | (HR @ 2 mmol) - | (HR @ 1.9 mmol) - | (HR @ 1.8 mmol) - |
| heart rate | 4 beats/min | 5 beats/min | 6 beats/min |
| (SMART Zone min) | | | |

In an exemplary embodiment, if an individual completes the low test protocol, then the optimal heart rate range for the individual would be the individual's measured or calculated heart rate at LT1, 2.0 mmol lactate, minus 4 beats per minute. In another exemplary embodiment, if an individual completes the middle test protocol, then the optimal heart rate range for the individual would be the individual's measured or calculated heart rate at LT1, 1.9 mmol lactate, minus 5 beats per minute. In another exemplary embodiment, if an individual completes the high test protocol, then the optimal heart rate range for the individual would be the individual's measured or calculated heart rate at LT1, 1.8 mmol lactate, minus 6 beats per minute. A narrower heart rate range for an individual performing a low testing protocol is consistent with the shorter duration of fat oxidation and the lower power output (watts) that the individual can produce. In contrast, a broader heart rate range for an individual performing a middle testing protocol or a high testing protocol is consistent with the relatively longer durations of fat oxidation and the higher power output (watts) that the individual can produce.

In one embodiment, the calculated SMART Zone and the ability of an individual to work within the targeted zone is one component of training the individual's cardiometabolic response. As the individual is consistently able to work and train his or her body at the given steady state heart rates, i.e., SMART Zone, the individual's body will improve its lactate clearance capacity, improve its ability to increase fat oxidation, and produce higher power outputs at lower heart rates.

The individual's calculated SMART Zone, based on the data collected during the cardiometabolic testing protocol, may be one of the exercise protocols available to continue to monitor and track cardiorespiratory training after the test. In one embodiment, an individual having minimally sufficient cardiorespiratory health may receive SMART Zone heart rates to use during his or her exercise sessions. In some embodiments, an individual that is severely deconditioned as denoted by the inability to complete a test up to the designated lactate level may not receive a SMART training zone as the incorporation of minimal amounts of movement may be beneficial to the deconditioned individual.

In one embodiment, the individual's assigned exercise protocols, e.g., SMART zone and intervals, are available for download to the user application upon completion of the testing protocol and prior to the completion of the post-test recovery period. In one embodiment, the exercise protocols are available in the user application by the 3^{rd} minute of the post-test recovery allowing the individual to complete the post-test recovery in his or her newly assigned steady state zone.

### Interval Training

In addition to doing an activity at a consistent intensity for the duration of the exercise period, training in intervals is also known to be beneficial to the body. Specifically, interval training can also be used as a tool to help improve the cardiometabolic fitness of an individual. Interval training allows an individual to alternate between periods of higher intensity activity and lower intensity activity. In one embodiment, the higher intensity activity is performed for a shorter duration of time than the lower intensity activity. In an exemplary embodiment, an individual may alternate between a 1-second duration of a designated higher intensity heart rate zone and 3-second duration of lower activity. The time it takes to elevate the heart rate from the steady state zone to the interval zone may vary depending on effort applied, fitness level, and current fatigue level. Similarly, the heart rate recovery from the interval zone to the steady state zone may vary depending on fitness level and fatigue level. In one embodiment, the periods of higher intensity activity and lower intensity activity may be customized to account for various individual needs and fitness levels.

As an individual's cardiometabolic fitness improves, he or she can exercise for longer periods of time and with greater intensity. In various embodiments, various interval heart rates may be available to individuals based on the specific needs of the individual and as evidenced by the data observed during the testing protocol. In one embodiment, an individual may be provided one or more interval training heart zones. In another embodiment, an individual may be provided a higher interval heart rate zone above the steady state heart rate zone that also includes access to all of the lower interval heart rate zones above the steady state heart rate zone. In another exemplary embodiment, if an individual is provided a high intensity interval training (HIIT) zone, they may also be provided with a moderate intensity interval training (MIIT) zone. In another exemplary embodiment, if an individual is provided a sprint intensity interval training (SIIT) zone, they may also be provided with a HIIT zone and a MIIT zone.

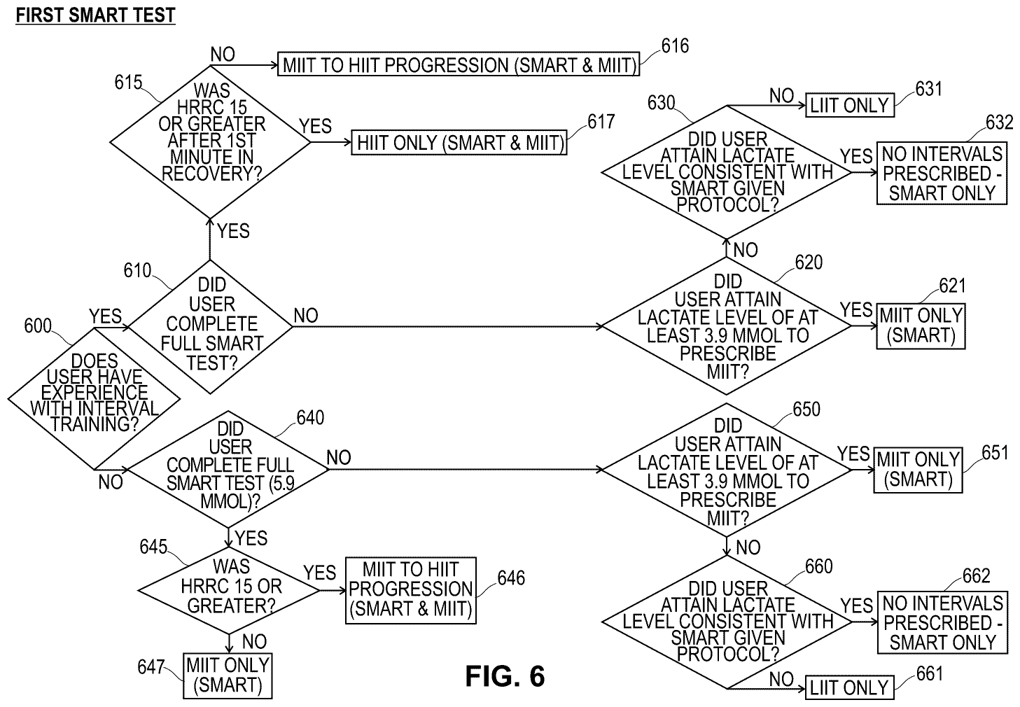

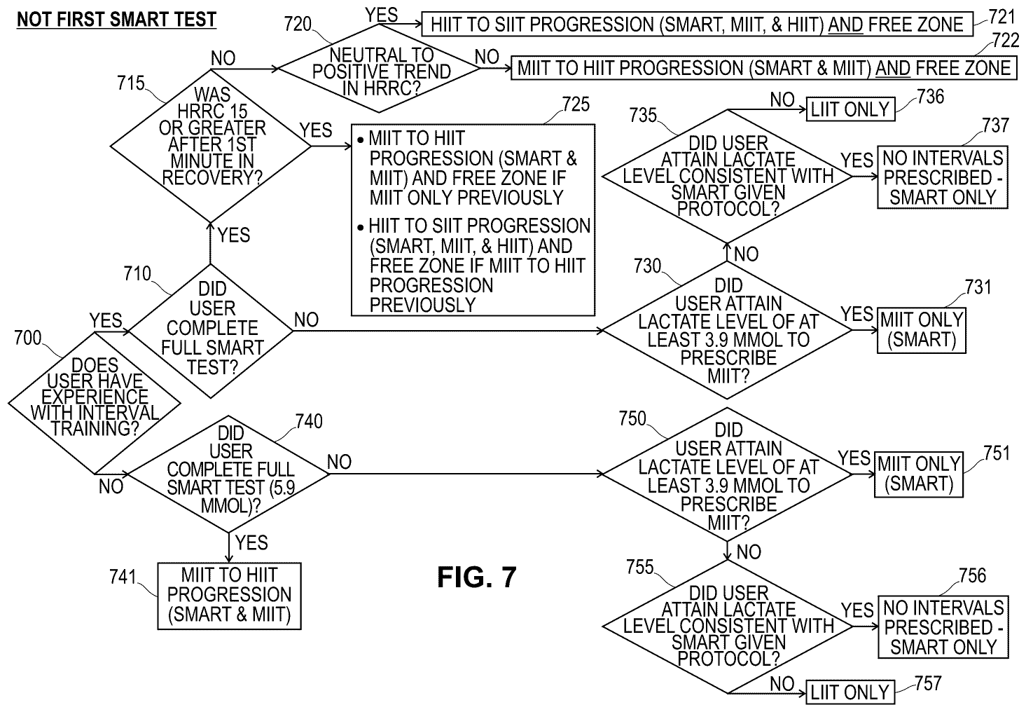

Specifically, in accordance with the methods of the present application, the interval levels (e.g., MIIT, HIIT, SIIT) and the associated training heart rates are assigned based on the behavior of the individual's unique biology and current level of health and fitness as measured by the data collected in the pre-test assessment and during the testing protocol. This is a significant improvement over previous zone training methods in which, at best, appropriate training zones for individuals were determined as a function of basic demographic data such as age without any consideration for an individual's level of health or fitness. In the present application, intervals are only assigned to an individual after the completion of the testing protocol and are determined based on multiple factors, specific to the individual, including whether or not he or she has previously completed a testing protocol or has previous experience with interval training. Because the assignment of interval training protocols, and any associated progressions or regressions, are based on the specific needs of the individual, this makes the interval training programs of the present application safer, more effective in achieving measurable health and fitness benefits, and more conducive to long-term adherence. FIGS. 6-7 illustrate an exemplary flow for determining the intervals that are assigned. FIG. 6 illustrates factors for consideration if the person is completing the first SMART Test protocol. Some users have experience with interval training while others do not 600. If the user has experience with interval training and completed the entire test 610, the user's heart rate recovery at the first minute after the test ends will be used to determine the assigned intervals. If the heart rate recovery at the first minute is 15 bpm or greater, the user will be assigned a HIIT interval for training 617. If the heart rate recovery was less than 15 bpm in the first minute 615, the user will be assigned a MIIT to HIIT progression 616. If the user did not complete the full SMART Test 610, the user's lactate samples will be evaluated to see if the user attained a level of at least 3.9 mmol 620. If the user attained at least 3.9 mmol, the user will be assigned a MIIT interval for training 621. If the user did not attain a lactate level of at least 3.9 mmol 620, the user's lactate reached a level consistent with a steady state level given the assigned testing protocol 630. If the user did not achieve a minimum lactate level consistent with steady state, LIIT is assigned 631. If the user achieved a minimum lactate level consistent with steady state, the user is only assigned the steady state zone for training 622.

If the user does not have experience with interval training 600 the user may be assigned different intervals based on the output from the testing protocol. If the user did not complete the full SMART Test 640, the user's lactate samples will be evaluated to see if the user attained a level of at least 3.9 mmol 650. If the user attained at least 3.9 mmol, the user will be assigned a MIIT interval for training 651. If the user did not attain a lactate level of at least 3.9 mmol 650, the user's lactate reached a level consistent with a steady state level given the assigned testing protocol 660. If the user did not achieve a minimum lactate level consistent with steady state, LIIT is assigned 661. If the user achieved a minimum If the user is not familiar with intervals, but completed the entire test 640, and the user's heart rate recovery at the first minute after the test ends is 15 bpm or greater 645, the user will be assigned a MIIT to HIIT progression 646. If the heart rate recovery was less than 15 bpm in the first minute 645, the user will be assigned a MIIT interval for training 647. If the lactate level consistent with steady state, the user is only assigned the steady state zone for training 662.

After the first SMART Test, a different assessment is used to determine the interval assignment. FIG. 7 illustrates factors for consideration if the person has previously completed a SMART Test protocol. If the user has experience with interval training 700 and completed the entire test 710, the user's heart rate recovery at the first minute after the test ends will be used to determine the assigned intervals. If the heart rate recovery at the first minute is 15 bpm or greater 715, the user will either be assigned a MIIT to HIIT progression and Free Zone (if previously assigned MIIT only) or HIIT to SIIT progression and Free Zone (if previously assigned MIIT to HIIT progression) interval for training 725. If the heart rate recovery was less than 15 bpm in the first minute 715, the user's heart rate recovery trend will be analyzed 720. The trend is determined by the weekly average heart rate recovery over the most recent three weeks of using the interval training protocol. If the recovery rate is increasing beyond a 6 beat range, then it is a positive trend, if it is decreasing beyond a 6 beat range, then it is a negative trend. If the heart rate appears within a 5 beat range up or down, then it is a neutral trend. If the heart rate recovery trend is determined to be neutral or positive, the user will be assigned HIIT to SIIT progression and Free Zone 721. If the heart rate recovery trend is determined to not be neutral or positive, the user will be assigned MIIT to HIIT progression and Free Zone 722. If the user has experience with interval training 700, but did not complete the full SMART Test 710, the user's lactate samples will be evaluated to see if the user attained a level of at least 3.9 mmol 730. If the user attained at least 3.9 mmol, the user will be assigned a MIIT interval for training 731. If the user did not attain a lactate level of at least 3.9 mmol 730, the user's lactate reached a level consistent with a steady state level given the assigned testing protocol 735. If the user did not achieve a minimum lactate level consistent with steady state, LIIT is assigned 736. If the user achieved a minimum lactate level consistent with steady state, the user is only assigned the steady state zone for training 737.

If the user does not have experience with interval training 700, but completed the entire test 740, the user will be assigned a MIIT to HIIT progression for training 741. If the user did not complete the full SMART Test 740, the user's lactate samples will be evaluated to see if the user attained a level of at least 3.9 mmol 750. If the user attained at least 3.9 mmol, the user will be assigned a MIIT interval for training 751. If the user did not attain a lactate level of at least 3.9 mmol 750, the user's lactate reached a level consistent with a steady state level given the assigned testing protocol 755. If the user did not achieve a minimum lactate level consistent with steady state, LIIT is assigned 751. If the user achieved a minimum lactate level consistent with steady state, the user is only assigned the steady state zone for training 756.

### Interval Training Examples

Example 1: Prior to the start of the first SMART Test, the individual reports not having any prior experience with interval training. This information is recorded in the device or system in conjunction with all other pre-test data points collected. Based on the pre-test data and the individual's performance at the initial testing protocol, the individual is assigned a low test protocol for completing the test. The individual progresses through the test and the heart rate data, lactate measurements, and power outputs are measured and input into the device or system at the assigned intervals. The individual completes the low protocol test and after the first minute of recovery, has a heart rate recovery rate of 24 bpm. This individual would be assigned to MIIT to HIIT interval progression, including the SMART Zone and MIIT training zones.

Example 2: Prior to the start of the first SMART Test, the individual reports having prior experience with interval training. This information is recorded in the device or system in conjunction with all other pre-test data points collected. Based on the pre-test data and the individual's performance at the initial testing protocol, the individual is assigned a middle test protocol for completing the test. The individual progresses through the test and the heart rate data, lactate measurements, and power outputs are measured and input into the device or system at the assigned intervals. The individual did not complete the middle protocol test but attained a lactate level of 2.3 mmol before terminating the test. This individual achieved a lactate measurement greater than the minimum 1.9 mmol at the middle protocol required for SMART Zone and therefore would be assigned a SMART Zone protocol. This individual would not be assigned any additional intervals, such as MIIT or HIIT at this time.

Example 3: Prior to the start of the second SMART Test, the individual reports not having any prior experience with interval training. This information is recorded in the device or system in conjunction with all other pre-test data points collected. Based on the pre-test data and the individual's performance at the initial testing protocol, the individual is assigned a middle test protocol for completing the test. The individual progresses through the test and the heart rate data, lactate measurements, and power outputs are measured and input into the device or system at the assigned intervals. The individual did not complete the middle protocol test but attained a lactate level of 4.1 mmol before terminating the test. This individual achieved a lactate measurement greater than the minimum 3.9 mmol required for MIIT and therefore would be assigned a MIIT interval, in addition to the SMART Zone protocol. This individual would not be assigned any additional intervals, such as MIIT to HIIT progression or HIIT at this time.

Example 4: Prior to the start of the second SMART Test, the individual reports having prior experience with interval training. This information is recorded in the device or system in conjunction with all other pre-test data points collected. Based on the pre-test data and the individual's performance at the initial testing protocol, the individual is assigned a high test protocol for completing the test. The individual progresses through the test and the heart rate data, lactate measurements, and power outputs are measured and input into the device or system at the assigned intervals. The individual completes the high protocol test and after the first minute of recovery, has a heart rate recovery rate of 14 bpm with a heart rate recovery trend that shows an overall trend of improvement in recovery. This individual would be assigned to HIIT to SIIT interval progression, including the SMART Zone, MIIT, and HIIT training zones as well as the Free Zone exercise zone.

The following paragraphs provide additional details regarding each of the intervals and the interval progressions. In each of the training zones described below, the heart rate is continuously measured via a heart rate monitor and transmitted to a device or system to ensure that the user is exercising in the designated training zone and for the appropriate amount of time. In an exemplary embodiment, if the user's heart rate falls below the designated zone, the user may receive an audio or visual alert. Similarly, if the user's heart exceeds the designated training zone, the user may receive an audio or visual alert. Based on the user's heart rate and time spent in a training zone, the user may also be alerted to increase his or her heart rate or decrease his or her heart rate to reach a new training zone (e.g., if the user is working in SMART Zone, he or she may be prompted to increase heart rate to HIIT zone).

### LIIT: Light Intensity Interval Training

The light intensity interval training zone offers a heart rate training zone that supports safer exercise training for severely deconditioned and sedentary users. An individual assigned to exercise in this zone may have difficulty maintaining the steady state zone that he or she was assigned or alternatively, may not have been assigned a steady state zone due to an inability to, or difficulty in, completing the assigned testing protocol. The LIIT zone offers a heart rate interval training zone that begins at or near the resting heart and no higher than the steady state training zone.

In the LIIT zone, a user may begin exercising at his or her resting heart rate and increase intensity of physical activity to reach the SMART zone heart rate, and then return the heart rate back or near to a resting heart rate. The repetition of the increase in heart rate to SMART zone and the return to a resting heart rate throughout the session or throughout the day can be accomplished with non-exercise physical activity (e.g., walking). As an individual's capacity to sustain or tolerate physical activity over longer periods of time increases, he or she may be assigned a sustained steady state and higher heart rate training intervals.

### MIIT: Moderate Intensity Interval Training

The moderate intensity interval training is a training zone that may also be provided to a person that is physically deconditioned, not accustomed to exercise or interval training, not accustomed to higher intensity activities, or may have had difficulties in completing the assigned testing protocol. The MIIT zone may help an individual learn to perform and tolerate interval training. The MIIT zone offers a heart rate training zone that is higher than the steady state training zone.

In the MIIT zone, a user may begin an interval session at his or her resting heart rate and then increase his or her heart rate to the steady state SMART zone. In one embodiment, the individual may work in the steady state heart rate zone for a defined duration before increasing the heart rate to the defined MIIT zone. The individual may work at the MIIT zone for a for a defined duration before returning the heart rate back to the steady state heart rate zone. In one embodiment, an individual will continue to alternate between the steady state zone and the MIIT zone for a defined number of times and remain in each zone for the defined duration of time.

### HIIT: High Intensity Interval Training

The high intensity interval training is a training zone that may be provided to a person that can successfully complete the assigned testing protocol and may have experience with interval training. The heart rates provided for the HIIT zone are higher than the MIIT zone heart rates.

In the HIIT zone, a user may begin an interval session at his or her resting heart rate and then increase his or her heart rate to the steady state SMART zone. In one embodiment, the individual may work in the steady state heart rate zone for a defined duration before increasing the heart rate to the defined HIIT zone. The individual may work at the HIIT zone for a for a defined duration before returning the heart rate back to the steady state heart rate zone. In one embodiment, an individual will continue to alternate between the steady state zone and the HIIT zone for a defined number of times and remain in each zone for the defined duration of time.

### SIIT: Sprint Intensity Interval Training

The sprint intensity interval training is a training zone that may be provided to a person that shows that the individual has a high-level of cardiometabolic fitness. The heart rates provided for the SIIT zone are higher than the HIIT zone heart rates.

In SIIT, an individual works at a maximum level or full effort, such as during a full-out sprint. In the SIIT zone, a user may begin an interval session at his or her resting heart rate and then increase his or her heart rate to the steady state SMART zone. In one embodiment, the individual may work in the steady state heart rate zone for a defined duration before increasing the heart rate to the defined SIIT zone. The individual may work at the SIIT zone for a for a defined duration before returning the heart rate back to the steady state heart rate zone. In one embodiment, an individual will continue to alternate between the steady state zone and the SIIT zone for a defined number of times and remain in each zone for the defined duration of time.

The transitions between the steady state SMART zone heart rates and each of the MIIT, HIIT, or SIIT interval zones represent the transitions between periods of higher fat oxidation levels and lower fat oxidation levels. These intervals also represent the transitioning between LT1 and LT2, or maximal lactate steady state, stimulating the body to accumulate lactate and then remove lactate. The heart rate recovery rate, or HRRC, is also tracked during each of the MIIT, HIIT, and SIIT interval training sessions.

### Free Zone: Open Zone Training

The Free Zone is an open training protocol that does not direct the individual to work at any particular heart rate or for any set duration of time. Instead, the Free Zone allows the individual to exercise or work in whatever manner he or she chooses while tracking the individual's heart rate throughout the Free Zone session.

The individual is able to see all of the zones (e.g., LIIT, steady state, MIIT, HIIT, and SIIT) so the individual is able to see the zone they are working in and the duration they are working in each zone. This may be available to a user after they have spent some time working in the different zones and training the body to progress and regress the heart rate as prescribed. The Free Zone setting can be useful for circuit and/or cluster training, for monitoring during individual or group interval training with a variety of modalities including, but not limited to, most types of resistance training, treadmills, climbing, crawling, carrying, pushing, pulling, swimming, sprinting, etc. This setting is also useful as a diagnostic or observational tool to explore what the User's exercise stress response might be to any particular event or course such as a 10 k, a long weekend walk, hiking a trail with varied elevation, cycling, etc. relative to the metabolic thresholds determined at testing. HRRC is inactive within this setting.

### Exercise Monitoring and Adaptations

A user may be provided with various exercise protocols, including a steady state training zone and one or more interval training zones. In various embodiments, the steady state SMART zone provides the base protocol for the user to use for his or her training. This steady state zone also serves as the heart rate targets for the warmup and cooldown periods for the assigned interval training sessions MIIT, HIIT, or SIIT. While guidelines for recommended daily and weekly activity levels are provided by agencies such as the Center for Disease Control and Prevention, in an exemplary embodiment of the present application, the user may spend at least 10 min in SMART zone per session.

In various embodiments, the individual is provided a specific interval protocol based on the individual's performance during the testing protocol. As the individual exercises more, the body becomes more efficient working at the steady state heart rates and recovering after each interval at heart rates above the steady state. Accordingly, in various embodiments, each interval protocol may be progressed or regressed based on the individual, his or her consistency and adherence in performing the exercise protocols, and the adaptations of the individual's body to the program. In an exemplary embodiment, an individual may complete at least one complete interval training session (warmup, 6 or more intervals, each interval including increase heart rate to designated heart rate above steady state, followed by a return of heart rate to steady state, and cooldown) per week.

### MIIT Only

In one embodiment, an individual may be assigned a MIIT interval if he or she was able to attain a lactate concentration level of at least 3.9 mmol, even if he or she had difficulty in completing the testing protocol or was unable to complete the entire testing protocol. An individual with a MIIT only interval protocol may have the following interval training program provided, including the appropriate progression and regression logic based on his or her adherence.

| | Initial Interval | | |
|---|---|---|---|
| | Protocol | Progression | Regression |
| Intervals per | MIIT: 4 | Increase by 2 | For every one- |
| session | | intervals every | week period |
| | | 3 weeks if user: | the user does |
| | | Completes at | not complete |
| | | least one | at least one |
| | | full | interval, reduce |
| | | interval session | the number of |
| | | per week | MIIT intervals |
| | | Completes at | by 1. MIIT |
| | | least one full | intervals should |
| | | interval session | not be reduced |
| | | for 3 | below the |
| | | consecutive | minimum weekly |
| | | weeks | intervals (4 |
| | | | intervals/week) |
| Session | Min: 1 | Min: 2 | |
| frequency | session/week | sessions/week | |
| | Max: 3 | Max: 3 | |
| | sessions/week on | sessions/week on | |
| | non-consecutive | non-consecutive | |
| | days, i.e., at | days, i.e., at | |
| | least 24-hours | least 24-hours | |
| | between interval | between interval | |
| | training | training | |
| | sessions. | sessions. | |
| Warm-up | 5 min in steady | 5 min in steady | |
| | state | state | |
| Cool-down | 5 min in steady | 5 min in | |
| | state | steady | |
| | | state | |

### MIIT to HIIT Progression

In one embodiment, an individual who completed a full testing protocol, but may demonstrate a lower heart rate recovery, or an individual who does not have prior experience with using interval training, may be provided this interval protocol progression. This interval progression tool allows an individual to start training and to increase his or her heart rate to higher levels while also increasing his or her capacity to recover. This progressive protocol is provided to appropriately progress an individual to the higher HIIT interval levels while eventually eliminating training at the lower MIIT interval levels.

| | Initial Interval | | |
|---|---|---|---|
| | Protocol | Progression | Regression |
| Intervals per | MIIT: 2; HIIT: 4 | Increase by 2 | For every one- |
| session | | intervals every | week period the |
| | | 3 weeks if user: | user does not |
| | | Completes at | complete at |
| | | least one full | least one |
| | | interval session | interval, reduce |
| | | per week | the number of |
| | | Completes at | HIIT intervals |
| | | least one full | by 1. HIIT |
| | | interval session | intervals should |
| | | for 3 | not be reduced |
| | | consecutive | below the |
| | | weeks | minimum weekly |
| | | The sequence of | intervals (4 |
| | | progression is: | intervals/week) |
| | | MIIT: 2; HIIT: 4 | If the user has |
| | | MIIT: 1; HIIT: 6 | progressed out |
| | | HIIT: 8 | of MIIT warmups |
| | | HIIT: 10 | (e.g., |
| | | HIIT: 12 | HIIT 8) |
| | | | then regression |
| | | | will only |
| | | | indicate HIIT |
| | | | intervals and |
| | | | MIIT removed. |
| Session | Min: 1 | Min: 2 | |
| frequency | session/week | sessions /week | |
| | Max: 3 | Max: 3 | |
| | sessions/week on | sessions/week on | |
| | non-consecutive | non-consecutive | |
| | days, i.e., at | days, i.e., at | |
| | least 24-hours | least 24-hours | |
| | between interval | between interval | |
| | training | training | |
| | sessions. | sessions. | |
| Warm-up | 5 min in steady | 5 min in steady | |
| | state | state | |
| Cool-down | 5 min in steady | 5 min in | |
| | state | steady | |
| | | state | |

### HIIT Only

In one embodiment, an individual may be provided with a HIIT only interval protocol. The HIIT protocol and corresponding progressions and regressions may be appropriate for an individual that completed his or her first testing protocol, has experience with interval training, or may have demonstrated higher heart rate recovery.

| | Initial Interval | | |
|---|---|---|---|
| | Protocol | Progression | Regression |
| Intervals per | HIIT: 4 | Increase by 2 | For every one- |
| session | | intervals every | week period the |
| | | 3 weeks if user: | user does not |
| | | Completes at | complete at |
| | | least one full | least one |
| | | interval session | interval, reduce |
| | | per week | the number of |
| | | Completes at | HIIT intervals |
| | | least one full | by 1. HIIT |
| | | interval session | intervals should |
| | | for 3 | not be reduced |
| | | consecutive | below the |
| | | weeks | minimum weekly |
| | | The sequence of | intervals (4 |
| | | progression is: | intervals/week) |
| | | HIIT: 4 | |
| | | HIIT: 6 | |
| | | HIIT: 8 | |
| | | HIIT: 10 | |
| | | HIIT: 12 | |
| Session | Min: 1 | Min: 2 | |
| frequency | session/week | sessions/week | |
| | Max: 3 | Max: 3 | |
| | sessions/week on | sessions/week on | |
| | non-consecutive | non-consecutive | |
| | days, i.e., at | days, i.e., at | |
| | least 24-hours | least 24-hours | |
| | between interval | between interval | |
| | training | training | |
| | sessions. | sessions. | |
| Warm-up | 5 min in steady | 5 min in steady | |
| | state | state | |
| Cool-down | 5 min in steady | 5 min in | |
| | state | steady | |
| | | state | |

### HIIT to SIIT

In one embodiment, this protocol may be provided for an individual that has some experience with the testing and exercise protocols (i.e., this protocol may not be available to an individual after his or her first test), has experience with interval training and demonstrates a higher heart rate recovery. This interval progression tool allows an individual to start training and to increase his or her heart rate to higher levels while also increasing his or her capacity to recover. This progressive protocol is provided to appropriately progress an individual to the higher SIIT interval levels while eventually eliminating training at the lower HIIT interval levels.

| | Initial Interval | | |
|---|---|---|---|
| | Protocol | Progression | Regression |
| Intervals per | HIIT: 2; SIIT: 4 | Increase by 2 | For every one- |
| session | | intervals every | week period the |
| | | 3 weeks if user: | user does not |
| | | Completes at | complete at |
| | | least one full | least one |
| | | interval session | interval, reduce |
| | | per week | the number of |
| | | Completes at | SIIT intervals |
| | | least one full | by 1. SIIT |
| | | interval session | intervals should |
| | | for 3 | not be reduced |
| | | consecutive | below the |
| | | weeks | minimum weekly |
| | | The sequence of | intervals (4 |
| | | progression is: | intervals/week) |
| | | HIIT: 2; SIIT: 4 | If the user has |
| | | HIIT: 1; SIIT: 6 | progressed out |
| | | SIIT: 8 | HIIT warmups |
| | | SIIT: 10 | (e.g., SIIT 8) |
| | | SIIT: 12 | then regression |
| | | | will only |
| | | | indicate SIIT |
| | | | intervals and |
| | | | HIIT removed. |
| Session | Min: 1 | Min: 2 | |
| frequency | session/week | sessions/week | |
| | Max: 3 | Max: 3 | |
| | sessions/week on | sessions/week on | |
| | non-consecutive | non-consecutive | |
| | days, i.e., at | days, i.e., at | |
| | least 24-hours | least 24-hours | |
| | between interval | between interval | |
| | training | training | |
| | sessions. | sessions. | |
| Warm-up | 5 min in steady | 5 min in steady | |
| | state | state | |
| Cool-down | 5 min in steady | 5 min in | |
| | state | steady | |
| | | state | |

### SIIT Only

In one embodiment, an individual may be provided with a SIIT only interval protocol. The SIT protocol and corresponding progressions and regressions may be appropriate for an individual that has completed at least two testing protocols, has experience with interval training, and may have demonstrated higher heart rate recovery. The SI T interval training protocol is most effective for physically conditioned individuals with higher levels of cardiometabolic fitness.

| | Initial Interval | | |
|---|---|---|---|
| | Protocol | Progression | Regression |
| Intervals per | SIIT: 4 | Increase by 2 | For every one- |
| session | | intervals every | week period the |
| | | 3 weeks if user: | user does not |
| | | Completes at | complete at |
| | | least one full | least one |
| | | interval session | interval, reduce |
| | | per week | the number of |
| | | Completes at | SIIT intervals |
| | | least one full | by 1. SIIT |
| | | interval session | intervals should |
| | | for 3 | not be reduced |
| | | consecutive | below the |
| | | weeks | minimum weekly |
| | | The sequence of | intervals (4 |
| | | progression is: | intervals/week) |
| | | SIIT: 4 | |
| | | SIIT: 6 | |
| | | SIIT: 8 | |
| | | SIIT: 10 | |
| | | SIIT: 12 | |
| Session | Min: 1 | Min: 2 | |
| frequency | session/week | sessions/week | |
| | Max: 3 | Max: 3 | |
| | sessions/week on | sessions/week on | |
| | non-consecutive | non-consecutive | |
| | days, i.e., at | days, i.e., at | |
| | least 24-hours | least 24-hours | |
| | between interval | between interval | |
| | training | training | |
| | sessions. | sessions. | |
| Warm-up | 5 min in steady | 5 min in steady | |
| | state | state | |
| Cool-down | 5 min in steady | 5 min in | |
| | state | steady | |
| | | state | |

In one embodiment, individuals will be provided multiple interval protocols. If the results of the test indicate that the user is only able to work at a steady state, then the user will be given the SMART Zone. If the test results indicate that the user can work at MIIT, then the user will be given SMART and MIIT zones. If the test results indicate that the user can work at HIIT, the user will be given HIIT, SMART, MIIT, and HIIT. If the test results indicate that the user can work at SIIT, then the user will be given SMART, MIIT, HIIT, and SIIT. If the test results indicate that the user can work at MIIT to HIIT progression, then the user will be given SMART, MIIT, and eventually HIIT. If the test results indicate that the user can work at HIIT to SIIT progression, then the user will be given SMART, MIIT, HIIT, and eventually SIIT.

### Interval Training Progression and Regression Examples

Example 1: After completing the testing protocol, a user was assigned a MIIT to HIIT progressive interval training protocol. Initially, this protocol provides that the individual would complete at least 1 interval training session, consisting of two (2) MIIT intervals and four (4) HIIT intervals, per week. After completing 1 interval session the first week, 2 interval sessions the second week, and 1 interval session the third week, the individual's MIIT to HIIT progressive interval training protocol was progressed. The new interval training protocol requires the individual to complete at least 2 interval training sessions, consisting of one (1) MIIT and six (6) HIIT intervals, per week. The individual would continue progression or regression based on his or her adherence to the interval training program.

Example 2: After completing the testing protocol, a user was assigned a HIIT only interval training protocol. Initially, this protocol provides that the individual would complete at least 1 interval training session, consisting four (4) HIIT intervals, per week. After consistently completing the required intervals to progress to a HIIT only interval training protocol requiring the individual to complete at least 2 interval training sessions, consisting of eight (8) HIIT intervals, per week, the individual is unable to complete at least one (1) interval training session for two consecutive weeks. In this case, the individual's HIIT only protocol would be reduced to at least 2 interval training sessions, consisting of six (6) HIIT intervals, per week.

Example 3: After completing the testing protocol, a user was assigned a HIIT to SIIT progressive interval training protocol. Initially, this protocol provides that the individual would complete at least 1 interval training session, consisting of two (2) HIIT intervals and four (4) SIIT intervals, per week. After completing 1 interval session the first week, 0 interval sessions the second week, and 1 interval session the third week, and continuing to complete only one interval session every other week. This individual would neither progress nor regress, but would remain at the current minimum requirements of at least 1 interval training session, consisting of two (2) HIIT intervals and four (4) SIIT intervals, per week until achieved consistently to allow for progression.

Adaptive Interval Regressions based on Heart Rate Recovery Monitoring

The ability of the heart rate to recover after a period of exercise is an important indicator of cardiovascular health. In an embodiment of the present application, heart rate recovery rate is another metric that is tracked to promote a safer training protocol, especially for more deconditioned individuals. Throughout the interval training session, the individual's heart rate is continuously monitored using a heart rate monitor. The heart rate data is continuously monitored and transmitted to a connected device or system not only to ensure that the individual is working at the designated training zone, including with the assistance of audio or visual cues to direct the user to increase or decrease heart rate, but also to accurately track the heart rate recovery as the individual recovers from a period of higher intensity exercise to a period of lower intensity exercise. In various embodiments, the higher the heart rate recovery rate, the more conditioned the cardiometabolic response.

In one embodiment, if during the performance of the interval sessions, the individual's heart rate recovery falls below 15 beats per minute for two (2) consecutive intervals, then the interval training for the session will be regressed to the next lower interval training zone to complete the interval training session. In various embodiments, if the individual is completing SIIT intervals, he or she may be regressed to HIIT; if the individual is completing HIIT intervals, he or she may be regressed to MIIT; and if the individual is completing MIIT intervals, he or she may be regressed to SMART zone. Alternatively, if the individual's heart rate continues to recover at a rate of 15 beats per minute or above, the individual will continue the training sessions in the same interval zone until the interval session is completed. In another embodiment, if the individual's heart rate recovery is below 15 for two (2) consecutive periods or continues to be below 15 for two (2) consecutive periods after regression, the individual will continue to regress until the individual either reaches the end of the interval session or the individual regresses to SMART zone. In a case where the individual regresses to SMART zone before the interval session is completed, the interval training session will terminate, and no other interval training sessions will be available on this day.

In various exemplary embodiments, the interval levels and heart rate recovery may be as follows:

Example 1

| Interval | 1 | 2 | 3 | 4 | 5 | 6 |
|---|---|---|---|---|---|---|
| Level | SIIT | SIIT | SIIT | SIIT | HIIT | HIIT |
| HRRC | 20 | 17 | 14 | 12 | 18 | 17 |

| Interval | 7 | 8 | 9 | 10 |
|---|---|---|---|---|
| Level | HIIT | HIIT | HIIT | HIIT |
| HRRC | 16 | 16 | 16 | 16 |

Example 2

| Interval | 1 | 2 | 3 | 4 | 5 | 6 | 7 | 8 | 9 | 10 |
|---|---|---|---|---|---|---|---|---|---|---|
| Level | HIIT | HIIT | HIIT | HIIT | MIIT | MIIT | MIIT | MIIT | SMART | SMART |
| HRRC | 20 | 15 | 14 | 13 | 13 | 16 | 12 | 12 | — | — |

Example 3

| Interval | 1 | 2 | 3 | 4 | 5 | 6 | 7 | 8 | 9 | 10 |
|---|---|---|---|---|---|---|---|---|---|---|
| Level | HIIT | HIIT | HIIT | HIIT | MIIT | MIIT | SMART | SMART | SMART | SMART |
| HRRC | 20 | 15 | 14 | 13 | 13 | 12 | — | — | — | — |

### Testing Feedback, Exercise Feedback, and Recalibration

In various embodiments, the ability to utilize specific user data to customize a protocol for the user and then to continuously monitor user biometrics and adapt the protocols are a component of the methods and systems described herein.

Testing Feedback: During testing process, the pre-test data inputs allow for the determination of an appropriate initial testing protocol based on the individual's current health and resting biometric data inputs. A feedback loop is created by testing user biometric data at various intervals and inputting such data into the testing algorithm, creating a testing protocol that is adapted in real-time, in response to the observed biometric data of the user. The adaptive feedback algorithms used during the testing protocol provide more accurate test result data and allow for more accurately tailored exercise protocols, e.g., SMART and interval zones, assigned for the individual to adhere to in between each testing session.

Exercise Feedback: The assigned interval training protocols are also adapted based on the user's weekly adherence to the protocol and the observed heart rate recovery behavior during each session. The ability to adapt the protocol to progress or regress the intensity or duration of the individual's exercise protocol is an important component to promoting a safer cardiometabolic training program and promoting long-term and sustainable cardiometabolic health and fitness benefits.

Recalibration: The present methods and systems allow for appropriately integrated feedback cycles of testing protocols-exercise protocols-recalibration testing protocols. This continuous cycle of testing using appropriately assigned testing protocols, followed by exercising using appropriately assigned exercise protocols, and then retesting allow for monitoring and capturing changes in the metabolic system resulting from exercise training. The consistent, gradual and incremental, acute to chronic exercise transitions encourage exercise consistency and avoids stagnation, and ultimately promotes sustainability and resilience for metabolic stress.

### System

The present system allows for communication between multiple, independent computing and processing devices. Each device is at least capable of receiving data inputs from multiple input components.

### Testing System

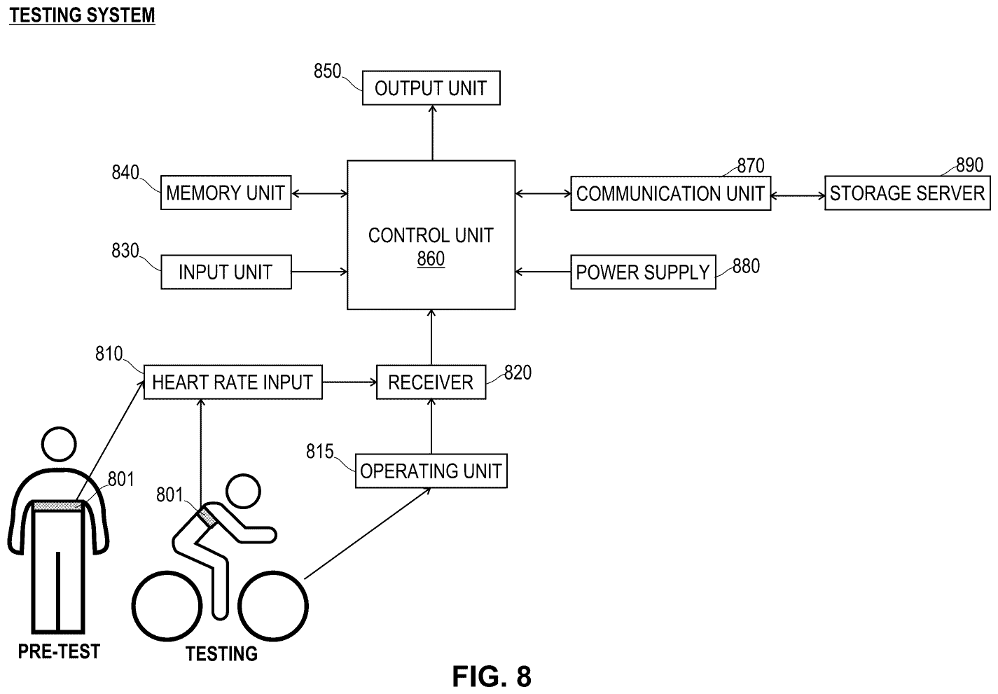

FIG. 8 illustrates one embodiment of the testing system. The testing system may comprise multiple connected components capable of receiving and transmitting data. In various embodiments a device such as a computer, tablet, or other mechanism capable of running or processing a computer program or application. It is understood that a computer, tablet, or other mechanism capable of running or processing a computer program or application may comprise various elements as illustrated in FIG. 8. Specifically, the device may be capable of allowing various data input including pre-test user response, or other information including alphanumeric inputs, photos, and various file uploads into the system using for example, a keypad or camera function on the device 830. In other exemplary embodiments, data may be wirelessly transmitted to a receiver 820. Biometric data, including heart rate 810 from a connected heart rate monitor 801 or data transmitted to the system from another connected component or system 815, such as an exercise bike, may be input into the system. The connected component may communicate with the device via local connection, wireless, or other remote network connection. The device also provides a means to process 860, store 840, and output information or cues visually via a display or audible via a speaker mechanism 850. Additionally, the device has a means for powering the system 880 and for communicating and transmitting data 870 between remote data servers 890.

In a specific embodiment, the device may house the algorithms necessary to process user data and determine sub-maximal testing protocols, including real-time progression and regression of testing protocol as set forth in the present application.

In one example, the testing system comprises a tablet with a testing application for administering the steps of the testing protocol. In one embodiment, the tester, or the person administering the testing protocol, provides power to the device and accesses the testing application or software program to begin a new test. The new test may begin with the tester accessing a scheduled appointment data or creating a new user appointment to begin the testing protocol for an individual.

In various embodiments, the tester application may be pre-populated with user information, such as completed health questionnaire data, medical clearance, participation waivers, stored or historical user health or biometric data, previous test results, exercise history, or any other such information or data relevant to the individual's health or the testing protocol. Such information may be populated from inputs provided via a user device having the user application (e.g., user smartphone or tablet), the user web portal accessible using for example a computer or internet-enabled device, or stored from previous testing appointments. In one embodiment, data may be input for a user by the tester at the start of the testing appointment, using the tester application on a device such as a tablet.

In one example the tester begins inputting data corresponding to the pre-test data required. The system may prompt the tester to manually input required metrics such as blood pressure, resting lactate, and medications for chronic health issues. Some data may be measured and input into the system manually while other data may be received from another device such as a heart rate monitor or other connected device. Once all the required pre-test data has been input to the system, the system processes the values according to the method and algorithms provided in the testing application and provides the prompts for the initial testing protocol via the device display. Throughout the initial testing protocol and the testing protocol, the system continues to receive various inputs directly from the tester via the tester device or from other connected devices, processes such data, and provides an appropriate output via the display.

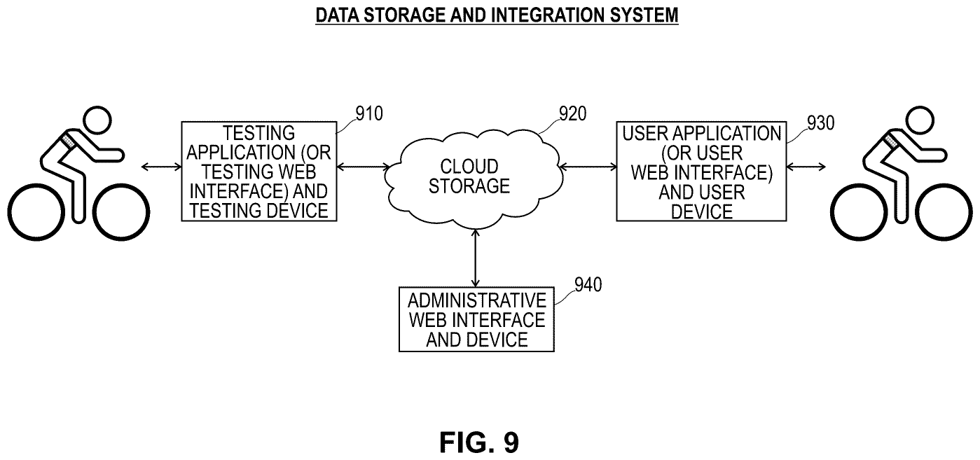

In one example, as shown in FIG. 9 at the conclusion of the testing protocol, all data input into the testing application 910, such as relevant user data, including pre-test data, data acquired during testing protocol, and the exercise protocol assigned to the user as a result of the testing process, are processed and transmitted for data storage. The data may be uploaded to a cloud storage 920 where it can be accessed from various authorized platforms, including downloaded to an authenticated user device or user web portal 930, a testing device or web portal 910, or an administrative portal 940. Data can be accessed or input from each authorized device or system.

### User System

One embodiment of the user system may comprise a device such as a computer, tablet, smartphone, or other mechanism capable of running or processing a computer program or application. The device is further capable of allowing for various data input including user registration data, exercise readiness data and other preliminary user responses, user biometric data or other information including alphanumeric inputs, photos, and various file uploads. The inputs may also be manually input into the system or transmitted to the system from a second connected component or system. The connected component may communicate with the device via local connection, wireless, or other remote network connection. The device also provides a means to process, store, and display data and information and may also provide audio output or cues.

The device may house the algorithms necessary to process user exercise data, track exercise protocol adherence, and determine protocol progressions and regressions. Specifically, real-time monitoring of biometric inputs may inform whether a regression is needed or exercise should be terminated for the user's safety.

In one example, the user system comprises a mobile phone with a user application for access to the user portal. In one embodiment, the user provides power to the device and accesses the user application to begin a session. The user may perform various tasks using the user application. In one embodiment the user may schedule a testing appointment using a calendar feature, said testing appointment may be uploaded to a cloud server and downloaded to an appropriate testing application or administrative portal. In various embodiments, the user may input data such as current weight, log nutrition intake or receive nutrition recommendations based on current nutrition input and health and fitness goals, make notes about health or fitness, or input any other data that may be prompted by the application or supported by the system. Additionally, a user may access previous test result data or exercise history, or access educational resources regarding health and fitness, including strength training guidance and interactive exercise sessions, or. In various embodiments the user application may allow for various means of two-communication such as voice and video sharing means. Such collaborative sessions may be useful in personal fitness training, physical therapy, and other body strengthening and rehabilitative purposes.

In one exemplary embodiment of the system, the user may open the user application, available on his or her mobile device. The user may manually log relevant data into the application or alternatively may input data by means of a connected device. In one example, after completing the testing protocol, the user may download the assigned exercise protocols from the shared storage and may store the exercise protocol on his or her local device or access it from the cloud. The user may access the application to begin an assigned exercise protocol, may see the exercise protocol on the user device display, and may also hear audio cues that prompt the user to take a certain action without visually seeing the display. The user may also connect a device such as a heart rate monitor or an exercise apparatus prior to beginning the exercise protocol. The visual display or the audio prompts may be used in combination or may be used independently to guide the user through the exercise training session. Relevant user biometric data may be transmitted directly to the application from a device such as a heart rate monitor, other wearable monitoring device, or an exercise apparatus. At the end of an exercise session, the user's performance data may be pushed or pulled to a shared cloud storage, for storage, analysis, and future use and recall.

### Tester Application

One component of the present application is the testing interface. The testing interface functions in various capacities to push information and data to the tester as well as to pull relevant data and information from various sources, including the user (e.g., user data, user input, user application data). In one embodiment, the tester may interface with the tester application program via an online web portal. In one embodiment, the tester may interface with the tester application program via an application on a device such a tablet. The application may be available on various development platforms such as iOS and Android and may be downloadable to a variety of devices such as a mobile phone, tablet, or other device capable of downloading and supporting applications.

In various embodiments the tester application may support a variety of functions including allowing a tester to input data or receiving data from other connected devices or input sources, storing data, processing data, and outputting data to tester and/or user. In an exemplary embodiment the tester application can provide specific and customized testing protocols that can be adapted in real-time as an individual user's test progresses.

In one embodiment, the tester application can send data to and receive data from a variety of sources. Various embodiments allow data to be received from cloud storage and other connected (remote, wireless, or local) devices or databases such as a user app or portal or a health monitoring device or biometric data input. The tester application may allow the tester to store data from the individual's test such as his or her user questionnaires, biometric inputs, notes, test performance data, aggregate reports, and other related information. The tester application may also allow access for the tester to retrieve historical data or access remote resources that may be relevant to the user's health or testing process.

### Pre-test Data Inputs

The test application can receive data from a variety of sources including data input by a user through the user portal or user application, data manually input by the tester, or data received via a device such as a heart rate monitor.

In various embodiments, the tester may complete a pre-test questionnaire with the user to ensure the individual is ready for and capable of physical activity. In various embodiments, the tester may be prompted to ask the user health screening questions to determine the user's state of readiness prior to testing. In various embodiments, the tester may be prompted to input measured or observed values such as the user's current height, weight, waist measurement, blood pressure, BMI, heart rate, pulse ox, etc. In various embodiments, various health and biometric data might be imported from a connected device such as a scale, heart rate monitor or other wearable, or a connected device capable of collecting relevant biometric or health data and transmitting such data to a program on a device or processing system capable of receiving the data.

### Protocol Assignment and Testing

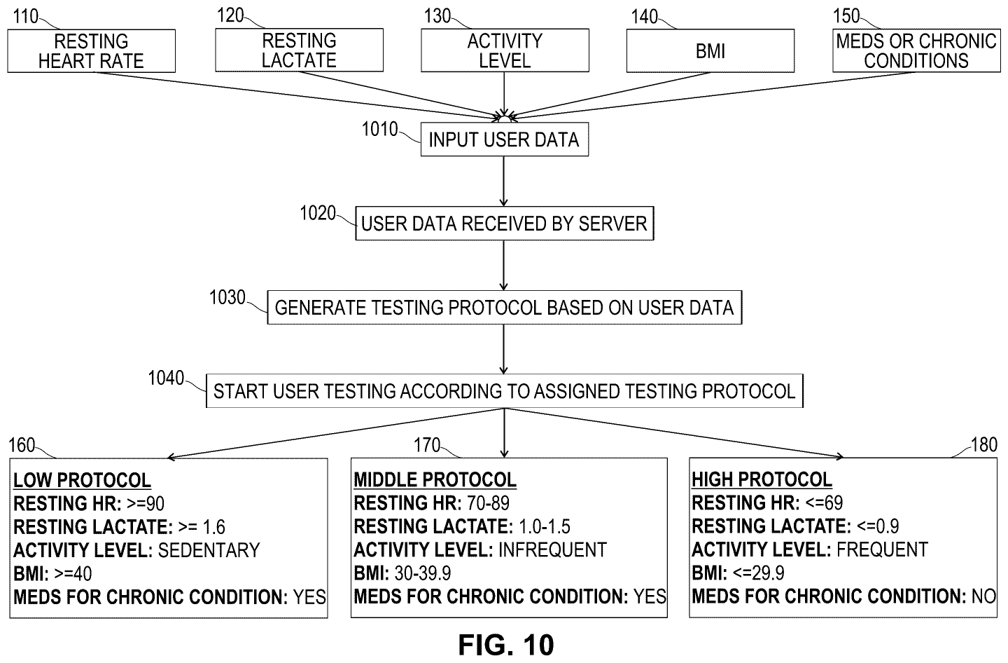

As shown in FIG. 10, the pre-test data such as user BMI 140, resting heart rate 110, resting lactate 120, data regarding chronic health issues and medications 150, and user activity level 130 are input into the tester application 1010 and received and processed 1020 according to various stored testing protocols. In various embodiments these various data points are used to determine an appropriate initial testing protocol 1030 for the individual to begin testing.

In various embodiments the tester guides the user to begin the assigned testing protocol 1040. A user may be assigned to an initial testing protocol of low 160, middle 170, or high 180, depending on the pre-test data inputs. In one embodiment data such as the individual's heart rate and lactate levels may continue to be collected and input into the testing application at periodic intervals during the testing process.

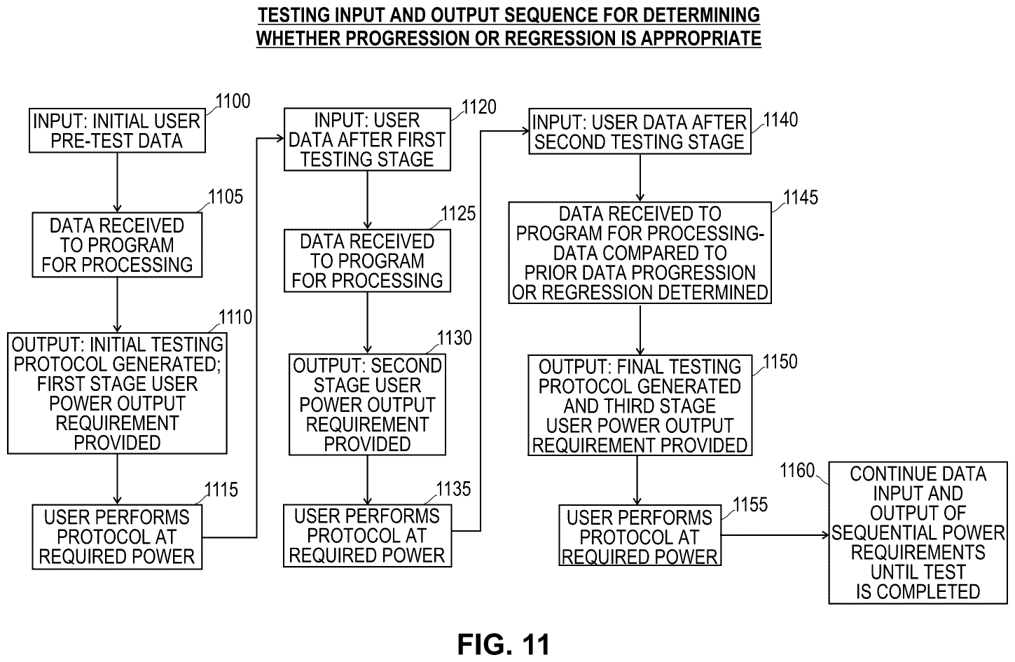

In various embodiment, the testing application may provide the tester with prompts such as a customized timer that can be used to track each interval period, alert the tester when an interval has been completed, and prompts or directs the tester as to what is the next step in the testing process or the next instruction that should be communicated to the user. In one embodiment the algorithms provided in the tester application determine whether the testing protocol should be progressed or regressed, in real-time, based on the data inputs received in the testing application during the testing protocol. In the embodiment of FIG. 11, pre-test data is input into the testing application 1100. The data is received and processed 1105 and the testing application provides an initial testing protocol 1110. The user is instructed to perform at the required power output for the initial testing protocol for the first testing interval 1115. At the completion of the first testing interval, user data is sampled and input into the testing application 1120. The data is received and processed 1125 and the testing application provides the user power output requirement for the second testing interval 1130. The user is instructed to perform at the required power output for the second testing interval 1135. At the completion of the second testing interval, user data is sampled and input into the testing application 1140. The data is received and processed 1145. The initial testing protocol (i.e., low, middle, or high) may be progressed, regressed, or unchanged based on an analysis of the pre-test data inputs and the inputs received after the first and second testing intervals. The testing protocol may be progress or regress after the completion of the second testing interval. The testing protocol assigned at the completion of the second testing interval is the testing protocol that user will complete the test at. The testing application provides the user power output requirement for the next testing interval, in accordance with the final testing protocol 1150. The user is instructed to perform at the required power output for the next testing interval 1155. The testing input and user output cycle continues until the test is completed 1160. The testing application may alert the tester to such changes in user's testing protocol. In one embodiment, the tester application may offer warnings, if the individual's biometric data exceeds certain defined thresholds (e.g., when blood pressure is too high to test, or when heart rate is too high to continue a test) or when the test should be terminated.

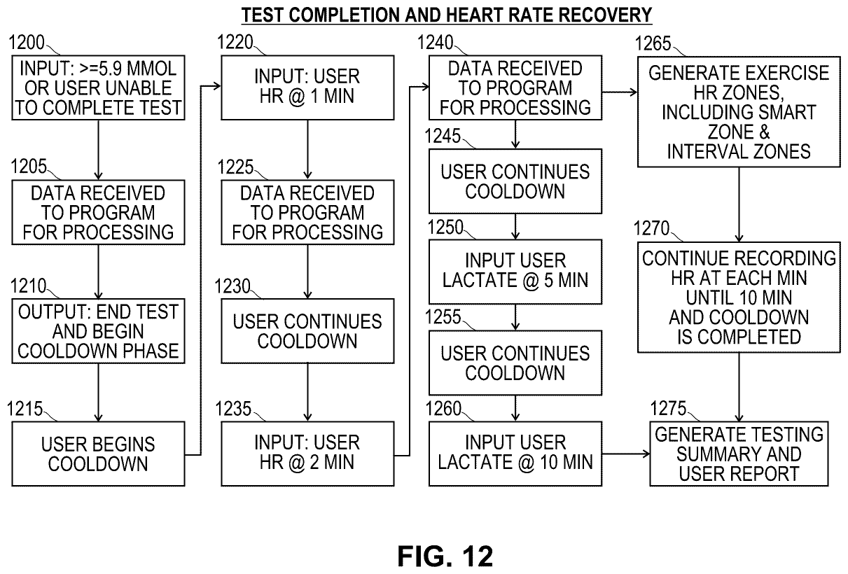

In various embodiments, the tester application also allows for data input and tracking during the post-test recovery process. The tester application algorithms may track heart rate recovery, various components of heart rate variability, and lactate clearance rate to allow for further user health analysis and more customized exercise protocols. FIG. 12 illustrates the data inputs observed at the end of the test and during the recovery process. If a user is either unable to complete the assigned testing protocol or the user measured biometric data reaches a level indicating that the test should be completed, the testing protocol is terminated 1200. This data is received by the testing application 1205 and the cooldown phase begins 1210. User is instructed to begin cooling down 1215, this process typically involves slowing or ceasing physical activity to allow the heart rate to begin recovering to a normal rate. The user's heart rate is measured after 1-minute 1220 and the heart rate is received into the program for processing 1225. The user continues the recovery process exerting minimal or no effort 1230. The user's heart rate is measured after 2-minutes 1235 and the heart rate is received into the program for processing 1240. After observing the heart rate recovery after 2 minutes, the user's specific target heart rate zones for steady state and for interval training is determined 1265. The user's heart rate continues to be measured and input into the system each minute until the cooldown period has ended 1270. As the user continues the recovery process exerting minimal effort, no effort, or working at steady state 1245 the user's blood lactate is sampled at 5 minutes and input into the system 1250. The user continues the cooldown period 1255 and the final blood lactate sample is taken at 10 minutes 1260. At the conclusion of the 10-minute cooldown period a final testing summary and report is generated 1275.

All data input into the testing application may be stored for future retrieval or transmitted to other authorized connected receiving devices, applications, or systems.

### User Application

One component of the present application is a user interface or portal. The user interface application functions in various capacities to push information and data to the user as well as to pull relevant data and information inputs from the user. In one embodiment, the user may interface with the program via an online web portal or the user may interface with the program via an application on a device, such as a mobile phone. The application may be available on various development platforms such as iOS and Android and may be downloadable to a variety of devices such as a mobile phone, tablet, or other device capable of downloading and supporting applications.

In various embodiments the user application may support a variety of functions including allowing user to input data, storing user data, processing user data, and outputting data to user. In various embodiments, the user application provides relevant health and fitness resources specific to the user. In an exemplary embodiment the application can track performance and guide the user through the completion of various exercise protocols (e.g., SMART Zone and interval training) that are customized to the user based on his or her unique combination of health data, fitness ability, and adherence to the recommended program and the individual's ability to adapt to the changing physical requirements of the assigned protocol(s).

### Biometric Data and User Inputs

In an embodiment, the user may input and record various data such as height, weight, BMI, or other such relevant health or biometric data. In another embodiment user data may be observed or collected from other sources. It is possible for data to be input from other connected devices such as a heart rate monitor, a wearable health track, or other such device. In another embodiment, the user app may either receive data pushed from a data source such as a cloud-based server or the user may pull data from such source. Data sources may be remote and connected through a wireless or virtual connections, or the data source may locally connected through a wireless or direct connection to the device housing the application.

The data from the user application is stored in a cloud storage housing the application and other related applications. In various embodiments, user data such as user reports, comparison reports showing progressions or regressions, exercise history, biometric data history, or various other information available in the application may be downloaded and stored locally to the user device or otherwise made available for use offline.

### Exercise Protocols and Exercise Tracking

In various embodiments, the user exercise protocols are downloaded from the cloud to the user device and accessible via the user application. The user may access the application and his or her assigned exercise protocols and be guided through an exercise session by way of visual and/or audio cues associated with the exercise protocol. The individual's performance data, such as heart rate and other biometric data trackable with use of, for example, a wearable device, or data collected from an exercise apparatus may be transmitted to the user application and used to guide the individual through the exercise protocol.

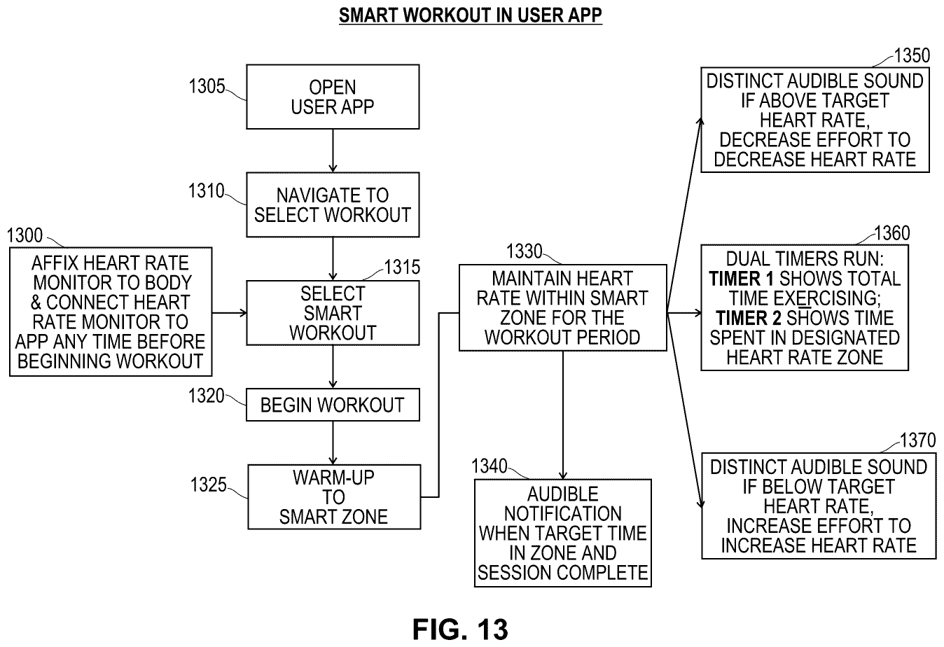

In a specific exemplary embodiment as shown in FIG. 13, a user may be guided through a steady state workout using the user application. In order to complete a workout, the user will open the user application 1305 and navigate in the user application 1310 to select the steady state workout 1315. The user will need to connect a heart rate monitor prior to beginning the workout 1300. Once the heart rate monitor is connected, the user will be able to begin the workout 1320. The user application will instruct the user to increase (or decrease) heart rate in order to reach the target steady state heart rate zone. Once the target heart rate is reached, user will be guided to maintain the heart rate within the heart rate zone for the desired workout period 1330. In various exemplary embodiments, an audible or visual notification may alert the user that his or her heart rate is above the target heart rate zone 1350 or may alert the user that his or her heart rate is below the target heart rate zone 1370. There may be at least two timing devices to track the workout. The first timer may track the total time exercising, while the second time may track the time spent in the target heart rate zone 1360. An audible or visual notification may also alert the user that the target time in heart rate zone has been reached 1340 and the workout may be ended or the workout may be extended for additional time.

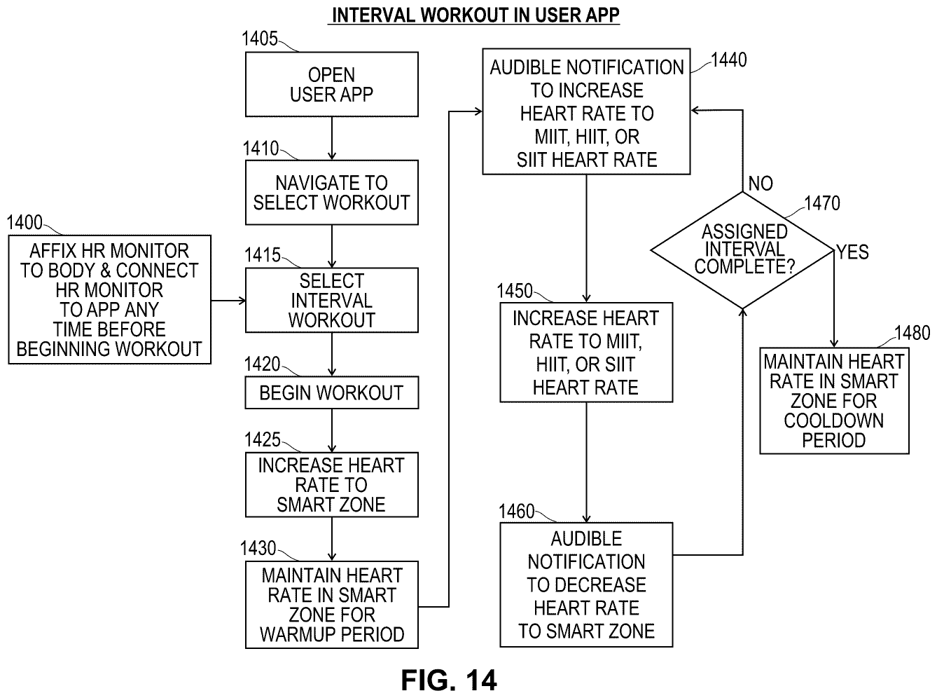

In another exemplary embodiment as shown in FIG. 14, a user may be guided through an interval workout using the user application. In order to complete a workout, the user will open the user application 1405 and navigate in the user application 1410 to select the interval workout 1415. The user will need to connect a heart rate monitor prior to beginning the workout 1400. Once the heart rate monitor is connected, the user will be able to begin the workout 1420. The user application will instruct the user to increase (or decrease) heart rate in order to reach the target steady state heart rate zone 1425. Once the target heart rate is reached, audible or visual notification may alert when user if he or she climbs above or falls below the target heart rate zone. The user will be guided to maintain the heart rate within the heart rate zone for the warmup period 1430. Once the warmup period is complete, an audible or visual notification will alert the user to increase the target heart rate to the designated interval training zone (e.g., MIIT, HIIT, or SIIT depending on the protocol) 1440. The user will increase output in order to increase heart rate to the designated heart rate zone 1450. Once the interval heart rate is reached an audible or visual notification will alert the user to decrease heart rate back to the steady state zone 1460. After reaching steady state for a set period, the system will check to see whether the user has completed the assigned training intervals 1470. If the user has not completed the assigned number of training intervals he or she may again be directed to increase his or her heart rate to the designated interval training zone 1440, increasing the heart rate to the interval training zone 1450, and then decreasing the heart rate back to steady state 1460. The cycle of increasing the heart rate to the interval training level and then decreasing the heart rate back to steady state is continued until the user has completed the assigned number of training intervals and is then directed maintain the heart rate in SMART Zone for cooldown 1480 to conclude the interval workout.

In one example, the application can receive inputs from the exercise sessions, track completed exercise sessions and protocol adherence, track current protocol adaptations such as progressions and regressions, and track historical data regarding the user (e.g., weight, BMI, etc.) and exercise protocol adherence and changes over time.

### Heart Rate Recovery and Heart Rate Variability

In one example, the user application monitors heart rate inputs in real-time during an exercise session. Specifically, heart rate is captured and transmitted to the application. The application may provide visual and/or audio cues to the user to adjust the current workload in order to increase or decrease the heart rate in accordance with the requirements of the exercise protocol. In one example, if a user has not reached a work output level necessary to reach the heart rate desired by the exercise protocol, the user may be prompted to increase the heart rate. In contrast, a user's real-time heart rate may indicate that the user is working too hard and should decrease the workload in order to decrease the heart rate.

Additionally, the heart rate may be tracked during interval training sessions. The real-time observation of the rate in which the heart rate recovers from the increased stress of the interval allows the algorithms of the user application to adjust the interval workout in real-time and even prompt the user to end the session if the user is not recovering from the stress of the workout at the expected rate.

In one embodiment, the ability of the user application to receive and process the user biometric data, including heart rate data, in real-time during a workout program also allows for other real-time adaptations to the workout. The ability to modify a workout in real-time allows a user to constantly exercise and train at an appropriate and effective level given the current behavior of the user's body systems. This data processing loop helps to keep the user engaged in the exercise protocols while also ensuring safe progressions for maximum results.

Nutrition tracking and Strength Training Guidance

In one example, the user application allows the user to log daily nutrition information such as meals, caloric intake, and macronutrient consumption. In one embodiment, the nutrition inputs may be analyzed in accordance with other health metrics such as fitness goals and caloric expenditure based on power output during tracked exercises, and appropriate recommendations made.

Additionally, in various embodiments, the user application provides resources to guide the individual through various strength training protocols or routines. The strength training may be through use of pre-recorded or on-demand sessions available through a resource library, real-time live group training, or live one-one training, accessible in the user application. The nutrition and strength training components are additional tools that are useful in improving the overall health and wellness of an individual user.

### Interactive Communities, Leader Tracking, and Exercise Incentives

In various embodiments, the user may engage with other users through the user app. In an exemplary embodiment, communities are available for users to connect to and support other users throughout their respective health and wellness journeys. Users may engage directly with other users, or post questions, comments, or relevant information to the community of other users. In one embodiment, there are multiple communities, each community with a different focus (e.g., healthy eating, weightlifting, motivation and goals, etc.). A member may select to become a member of one or more communities. In one embodiment there are both public communities that are open for anyone to join, and private communities where membership may only be available to select users (e.g., work group, family group, etc.).

In various embodiments, user exercise adherence data (e.g., how many minutes has user spent in exercise sessions, how many exercise sessions has user completed, etc.) is tracked over a given period and compared to other users. In one embodiment a user's performance may be compared only to other users within a specific group (e.g., work group, family group, etc.). In another embodiment, a user's performance may be compared to all other users. In various embodiments, user performance is ranked against the other users in the comparison set. In an exemplary embodiment, the ranking algorithm may consider metrics such as the number of minutes completed in the steady state SMART zone and the number of interval sessions completed in each given period. Based on the ranking algorithm and the performance and each user in a given period, users may appear in a rank-order listing comparing the performance of an individual user to the performance of other users within a specific grouping. In one embodiment, a user may be promoted or demoted in the rankings based on his or her performance in comparison to the performance of the other users in the group during the same period. In an exemplary embodiment, a ranking period may be for one week, one month, one quarter, etc., or in any combination thereof.

In various embodiments, uers may also be provided incentives based on the performance of exercises and adherence to the assigned exercise protocols. In one embodiment users may accumulate incentive rewards over multiple periods (e.g., weeks, months, quarters, etc.) and redeem them directly or through vendors, for reward items, gift cards, or cash equivalents, or rewards of comparable value.

### Other Features

In various other embodiments, numerous user-friendly features and educational resources can be conceived of and implemented within the spirit of the present disclosure. As an application focused on improving the health, fitness, and general wellness of the user through guided exercise protocols and educational tools and resources, many informational features such as video training or written materials may be available to the user through the user portal. Another embodiment may provide for the individual to use neurocardiac monitoring (e.g., using Heart Rate Variability or it's derivations) during testing as an input to the testing system and for use by the individual during the his or her exercise protocol as an input to the user system, as a direct or indirect assessment of the Autonomic Nervous System relative to the lactate/watts/heart rate relationships. Further, functionality to track other measurable biometric data such as blood pressure, glucose levels, A1C, lipids including HDL and LDL and numerous other data points relevant to cardiometabolic fitness and overall health are also conceived. Additionally, metrics such as sleep, steps, calorie intake (food tracker, including dietary recommendations based cumulative health data and goals), calorie expenditure (based on cumulative health data and tracked exercise), and women's health metrics may be tracked. Comprehensive data science and data management, as structured within this platform, may provide for a prognostic or predictive capacity of this platform to anticipate changes in future cardiometabolic health and fitness status. Data may be input manually by the user or tester, or collected from a connected device. Data may be uploaded from the user or tester apps or downloaded to the user or tester apps. User data may be protected with standard security protocols used to protect personal data.

The devices, systems and methods described herein can store, edit, and/or transmit data, including biometric data. The data can be stored in a database, such as for example a computer that has a processor configured to send the data to its memory and/or display it. The database and/or processor can comprise an input device (e.g., keyboard, touch screen, voice activation, biometric monitoring devices, fitness equipment, etc.) to allow a user to enter, display, edit, and/or transmit the data. The processor comprises logic to execute one or more instructions to carry instructions of the computer system. The logic for executing instructions may be encoded in one or more tangible media for execution by the processor. For example, the processor may execute codes stored in a computer-readable medium such as memory. The computer-readable medium may be, for example, electronic (e.g., RAM (random access memory), ROM (read-only memory), EPROM (erasable programmable read-only memory)), magnetic, optical (e.g., CD (compact disc), DVD (digital video disc)), electromagnetic, semiconductor technology, or any other suitable medium.

In various embodiments, an authorized administrator may input or edit data, approve or assign an exercise protocol based on the biometric data, exercise adherence data, or otherwise, for display at the user interface of the device or computer processor and/or another computer networked with computer processor. Although the components of the system are shown in some of the figures as separate, they may be combined in one or more computer systems. Indeed, they may be one or more hardware, software, or hybrid components residing in (or distributed among) one or more local or remote computer systems. It also should be readily apparent that the components of the system as described herein may be merely logical constructs or routines that are implemented as physical components combined or further separated into a variety of different components, sharing different resources (including processing units, memory, clock devices, software routines, logic commands, etc.) as required for the particular implementation of the embodiments disclosed. Indeed, even a single general purpose computer (or other processor-controlled device) executing a program stored on an article of manufacture (e.g., recording medium or other memory units) to produce the functionality referred to herein may be utilized to implement the illustrated embodiments. It also will be understood that a plurality of computers or servers can be used to allow the system to be a network based system having a plurality of computers linked to each other over the network or Internet or the plurality of computers can be connected to each other to transmit, edit, and receive data via cloud computers.

The computer (e.g., memory, processor, storage component, etc.) may be accessed by authorized users. The authorized user can interface with the computer via a user interface that may include one or more display devices (e.g., CRT, LCD, or other known displays) or other output devices (e.g., printer, etc.), and one or more input devices (e.g., keyboard, mouse, stylus, touch screen interface, smart phone, Apple Watch, or other known input mechanisms) for facilitating interaction of a user with the system via the user interface. The user interface may be directly coupled to database or directly coupled to a network server system via the Internet or cloud computing. In accordance with one embodiment, one or more user interfaces are provided as part of (or in conjunction with) the illustrated systems to permit users to interact with the systems.

The user interface device may be implemented as a graphical user interface (GUI) containing a display or the like, or may be a link to other user input/output devices known in the art. Individual ones of a plurality of devices (e.g., network/stand-alone computers, personal digital assistants (PDAs), WebTV (or other Internet-only) terminals, set-top boxes, cellular/phones, screenphones, pagers, blackberry, smart phones (e.g., iPhone, Android, etc.) iPad, Apple Watch, table, peer/non-peer technologies, kiosks, or other known (wired or wireless) communication devices, etc.) may similarly be used to execute one or more computer programs (e.g., universal Internet browser programs, dedicated interface programs, etc.) to allow users to interface with the systems in the manner described. Database hardware and software can be developed for access by users through personal computers, mainframes, and other processor-based devices. Users may access data stored locally on hard drives, CD-ROMs, stored on network storage devices through a local area network, or stored on remote database systems through one or more disparate network paths (e.g., the Internet).

The database for the systems and methods described herein can be stored in storage devices or systems (e.g., Random Access Memory (RAM), Read Only Memory (ROM), hard disk drive (HDD), floppy drive, zip drive, compact disk-ROM, DVD, bubble memory, flash drive, redundant array of independent disks (RAID), network accessible storage (NAS) systems, storage area network (SAN) systems, etc.), CAS (content addressed storage) may also be one or more memory devices embedded within a CPU, or shared with one or more of the other components, and may be deployed locally or remotely relative to one or more components interacting with the memory or one or more modules. The database may include data storage device, a collection component for collecting information from users or other computers into centralized database, a tracking component for tracking information received and entered, a search component to search information in the database or other databases, a receiving component to receive a specific query from a user interface, and an accessing component to access centralized database. The receiving component is programmed for receiving a specific query from one of a plurality of users. The database may also include a processing component for searching and processing received queries against data storage device containing a variety of information collected by collection device.

The disclosed system may, in some embodiments, be a computer network-based system. The computer network may take any wired/wireless form of known connective technology (e.g., corporate or individual LAN, enterprise WAN, intranet, Internet, Virtual Private Network (VPN), combinations of network systems, etc.) to allow a server to provide local/remote information and control data to/from other locations (e.g., other remote database servers, remote databases, network servers/user interfaces, etc.). In accordance with one embodiment, a network server may be serving one or more users over a collection of remote and disparate networks (e.g., Internet, intranet, VPN, cable, special high-speed ISDN lines, etc.). The network may comprise one or more interfaces (e.g., cards, adapters, ports) for receiving data, transmitting data to other network devices, and forwarding received data to internal components of the system (e.g., displays, smart phones, watches, etc.).

In accordance with one embodiment of the present application, the data may be downloaded in one or more textual/graphical formats (e.g., RTF, PDF, TIFF, JPEG, STL, XML, XDFL, TXT, power point, etc.), or set for alternative delivery to one or more specified locations (e.g., via e-mail, text, etc.) in any desired format (e.g., print, storage on electronic media and/or computer readable storage media such as CD-ROM, flash drive, etc.). The data of the current application can be stored in a computer readable data storage medium of a computer. Computer readable media, for example, store data that is accessible by a computer, such as magnetic cassettes, flash memory cards, digital video disks, Bernoulli cartridges, random access memories (RAMs), read only memories (ROMs), and the like and may also be used in an exemplary operating environment.

The devices, systems, and methods described herein may have a processor that can execute a computer model (e.g., a computer model having predictive capabilities that use artificial intelligence modeling techniques) that assign the biometric data a score used to predict one or more treatment plans. In some configurations, the biometric data that satisfies a threshold may be used by the processor to train an artificial intelligence model. Using a smaller training dataset, the processor can improve the training efficiency and timing.

While particular embodiments of the present disclosure have been shown and described, it will be appreciated by those skilled in the art that, based upon the teachings herein, changes and modifications may be made without departing from this disclosure and its broader aspects and, therefore, the appended claims are to encompass within their scope all such changes and modifications as are within the true spirit and scope of this disclosure. The true spirit and scope is considered to encompass devices and processes, unless specifically limited to distinguish from known subject matter, which provide equivalent functions as required for interaction with other elements of the claims and the scope is not considered limited to devices and functions currently in existence where future developments may supplant usage of currently available devices and processes yet provide the functioning required for interaction with other claim elements. Furthermore, it is to be understood that the disclosure is solely defined by the appended claims.

1. A method for testing the cardiometabolic fitness of a user, the method comprising:
collecting a pre-test biometric data and a pre-test health data from the user;
inputting collected the pre-test biometric data and the pre-test health data into a processing system, where the processing system processes the pre-test biometric data and the pre-test health data;
assigning a cardiometabolic test protocol, including an initial work output level, to the user based on the combination of pre-test biometric data and the pre-test health data;
instructing the user to begin the assigned initial cardiometabolic test protocol at the initial work output level for a defined interval period;
measuring the user a cardiometabolic test data at each defined interval period during the cardiometabolic test protocol, using one or more device capable of measuring the cardiometabolic test data;
inputting the measured cardiometabolic test data into the processing system at each defined interval, where the processing system processes the measured cardiometabolic test data and assigns the user a next work output level for each defined interval period based on the measured cardiometabolic test data;
instructing the user to increase the work output to the assigned next work output level for the defined interval period;
measuring the user cardiometabolic test data at each defined interval during the cardiometabolic test protocol, inputting the measured cardiometabolic test data into the processing system at each defined interval, where the processing system processes the measured cardiometabolic test data and assigns the user the next work output level based on the measured cardiometabolic test data, and repeating until the processing system processes the measured cardiometabolic test biometric data and does not assign a next workout output level, indicating the user has completed the cardiometabolic test protocol.

2. The method of claim 1, wherein collecting the pre-test biometric data includes at least one of resting heart rate or resting lactate, using a device capable of collecting resting heart or resting lactate, wherein the resting heart rate or resting lactate is collected immediately prior to beginning the cardiometabolic test.

3. The method of claim 1, wherein collecting the pre-test health data includes at least one of BMI, activity level, chronic medications, blood oxygen, or blood pressure, wherein the pre-test health data is collected immediately prior to beginning the cardiometabolic test.

4. The method of claim 1, wherein inputting the collected pre-test biometric and health data includes and the cardiometabolic test data is a computer system, such as a tablet or mobile device, capable of receiving data from manual input or other connected devices or systems, capable of processing the input data, and capable of sending data to other connected devices, including storage systems.

5. The method of claim 1, wherein measuring the user cardiometabolic test data at each defined interval during the cardiometabolic test protocol is done in a three-minute interval periods.

6. The method of claim 1, wherein assigning the cardiometabolic test protocol, including an initial work output level, may be either a low test protocol, with an initial work output level of 5 watts, a middle test protocol with an initial work output of 15 watts, or a high test protocol with an initial work output of 15 watts, and wherein the assigned cardiometabolic test protocol may be regressed, progressed, or remain the same, so that the cardiometabolic test protocol assigned to the user at the start of the cardiometabolic test protocol may be different from the cardiometabolic test protocol that the user completes the cardiometabolic test at.

7. The method of claim 1, wherein indicating the cardiometabolic test protocol is complete may occur when the measured cardiometabolic test data includes measuring lactate at each defined interval during the cardiometabolic test, and further wherein the measured lactate level reaches 5.9 mmol concentration.

8. A method of calculating cardiometabolic fitness of a user comprising:
measuring the BMI of the user, wherein the BMI comprises the individual's weight measured in kilograms and the individual's height measured in meters, such that the BMI is equal to the weight in kilograms divided by the height in meters squared;
inputting measured BMI into a processing system;
measuring a resting lactate concentration, wherein the resting lactate concentration is measured prior to physical activity;
inputting the measured resting lactate concentration into the processing system;
commencing physical activity at an assigned cardiometabolic fitness test protocol and increasing a power output of the physical activity over time as provided by the cardiometabolic fitness test protocol in combination with the measured a cardiometabolic test data, including heart rate and lactate, and wherein the power output of the physical activity is measured in watts;
measuring the power output in watts, heart rate and the lactate concentration at each set interval and inputting the data into the processing system;
processing the power output in watts, heart rate and the lactate concentration at each interval to identify the power output in watts at the first lactate threshold (LT1), providing a data output equal to the power output in watts at LT1, divided by the BMI and multiplied by a factor of 10.

9. A method of establishing cardiometabolic steady state heart rate zones, comprising:
testing the cardiometabolic fitness of a user at an assigned a cardiometabolic test protocol, wherein the cardiometabolic test protocol may be a low cardiometabolic test protocol, a middle cardiometabolic test protocol, or a high cardiometabolic test protocol, collecting cardiometabolic test data and inputting collected cardiometabolic test data into a connected processing system at each defined interval during the cardiometabolic test protocol until the user completes the cardiometabolic test protocol;
assigning a steady state zone heart rate zone based on the cardiometabolic test protocol the user completes and the cardiometabolic test data collected during the cardiometabolic test protocol and input into the connected processing system, and processed by the processing system.

10. The method of claim 9, wherein collecting cardiometabolic test data may include a heart rate, wherein the heart rate is collected using a device capable of measuring heart rate and transmitting measured heart rate to the connected processing system, and lactate data, wherein the lactate is collected using a device capable of measuring lactate and the lactate data is input into the connected processing system.

11. The method of claim 10, wherein assigning a steady state heart rate zone to the user completing the low cardiometabolic testing protocol consists of processing the user cardiometabolic test data, including lactate data, input into the connected processing system, measure when the cardiometabolic test biometric lactate is 2 mmol, wherein the cardiometabolic test biometric heart rate data at a cardiometabolic test biometric lactate measure of 2 mmol is the maximum steady state heart rate of the steady state heart rate zone and the cardiometabolic test biometric heart rate data at a cardiometabolic test biometric lactate measure of 2 mmol less 4 beats per minute is the minimum steady state heart rate of the steady state heart rate zone.

12. The method of claim 10, wherein the user completes the middle cardiometabolic testing protocol, the steady state training zone is calculated by observing the user cardiometabolic test biometric data heart rate at a lactate measure of 1.9 mmol, wherein the heart rate at lactate measure of 1.9 mmol is the maximum steady state heart rate and the heart rate at a lactate measure of 1.9 mmol less 5 beats per minute is the minimum steady state heart rate.

13. The method of claim 10, wherein the user completes the high cardiometabolic testing protocol, the steady state training zone is calculated by observing the user cardiometabolic test biometric data heart rate at a lactate measure of 1.8 mmol, wherein the heart rate at lactate measure of 1.8 mmol is the maximum steady state heart rate and the heart rate at a lactate measure of 1.8 mmol less 6 beats per minute is the minimum steady state heart rate.

14. A method of establishing cardiometabolic interval training zones, comprising: testing the cardiometabolic fitness of a user at an assigned a cardiometabolic test protocol, wherein the cardiometabolic test protocol may be a low cardiometabolic test protocol, a middle cardiometabolic test protocol, or a high cardiometabolic test protocol, collecting cardiometabolic test data and inputting collected cardiometabolic test data into a connected processing system at each defined interval during the cardiometabolic test protocol until the user completes the cardiometabolic test protocol;
assigning an interval training zone heart rate zone based on the cardiometabolic test protocol the user completes and the cardiometabolic test data collected during the cardiometabolic test protocol and input into the connected processing system, and processed by the processing system, wherein the interval training zone may include one or more of a light intensity interval training (LIIT), a moderate intensity interval training (MIIT), a high intensity interval training (HIIT), and a sprint intensity interval training (SIIT).

15. The method of claim 14, wherein collecting cardiometabolic test data may include a heart rate, wherein the heart rate is collected using a device capable of measuring heart rate and transmitting measured heart rate to the connected processing system, and lactate data, wherein the lactate is collected using a device capable of measuring lactate and the lactate data is input into the connected processing system.

16. The method of claim 14, wherein the MIIT interval training zone is determined by the heart rate at a lactate measure of 3.9 mmol, wherein the heart rate at lactate measure of 3.9 mmol is the maximum heart rate for the MIIT interval training zone and the heart rate at a lactate measure of 3.9 mmol less 4 beats per minute is the minimum heart rate for the MIIT interval training zone.

17. The method of claim 14, wherein the HIIT interval training zone is determined by the heart rate at a lactate measure of 5.9 mmol, wherein the heart rate at lactate measure of 5.9 mmol is the maximum heart rate for the HIIT interval training zone and the heart rate at a lactate measure of 5.9 mmol less 4 beats per minute is the minimum heart rate for the HIIT interval training zone.

18. The method of claim 14, wherein the SIIT interval training zone is determined by the heart rate at a lactate measure greater than 5.9 mmol, wherein the heart rate at lactate measure of greater than 5.9 mmol is the minimum heart rate for the SIIT interval training zone and the heart rate at a lactate measure greater than 5.9 mmol plus 4 beats per minute is the maximum heart rate for the SIIT interval training zone.

19-35. (canceled)
